(19) United States
(12) Patent Application Publication
(10) Pub. No.: US 2025/0154180 A1
(43) Pub. Date: May 15, 2025

(54) CRK-LIKE (CRKL) ADAPTOR PROTEIN INHIBITORS AND METHODS OF MAKING AND USING THE SAME

(71) Applicant: Rutgers, The State University of New Jersey, New Brunswick, NJ (US)

(72) Inventors: Dongfang Liu, Milburn, NJ (US); Xuening Wang, Old Bridge, NJ (US); Jacques Roberge, Princeton, NJ (US)

(73) Assignee: Rutgers, The State University of New Jersey, New Brunswick, NJ (US)

(21) Appl. No.: 18/834,952

(22) PCT Filed: Feb. 1, 2023
(86) PCT No.: PCT/US2023/012093

§ 371 Date: Jul. 31, 2024

Related U.S. Application Data
(60) Provisional application No. 63/305,354, filed on Feb. 1, 2022.

(51) Int. Cl. C07F 9/6574; A61K 31/352; A61K 31/39; A61K 31/665; A61P 35/00; C07D 493/04

(52) U.S. Cl. CPC C07F 9/65744; A61K 31/352; A61K 31/39; A61K 31/665; A61P 35/00; C07D 493/04

(57) ABSTRACT

Disclosed herein are embodiments of compounds that exhibit Crk-like protein inhibition. The compound embodiments can be used to suppress or prevent tumor cell growth and/or to inhibit a Crk-mediated signaling pathway. Methods of making and using the compound embodiments are disclosed herein.

### CROSS REFERENCE TO RELATED APPLICATION

This application claims the benefit of and priority to U.S. Provisional Patent Application No. 63/305,354, filed on Feb. 1, 2022, the entirety of which is incorporated herein by reference in its entirety.

### ACKNOWLEDGMENT OF GOVERNMENT SUPPORT

This invention was made with government support under grant number 1R01AI130197-01A1 awarded by the National Institutes of Health. The government has certain rights in the invention.

### FIELD

The present disclosure is directed to Crk-like adaptor protein inhibitors and methods of using the same.

### BACKGROUND

The Crk adaptor family of proteins plays critical roles in tumorigenesis, cancer metastasis, and immune responses. Phosphorylation of Crk is involved in tumor metastasis and natural killer (NK) cell inhibition and exhaustion. Theoretically, blocking phosphorylation of Crk (pCrk) in tumor cells and immune cells simultaneously would inhibit tumor growth and metastasis and restore effective immune responses by exhausted immune cells against tumors. Crk-like adaptor protein inhibitors are therefore needed in the art.

### SUMMARY

Disclosed herein are compound embodiments selected from

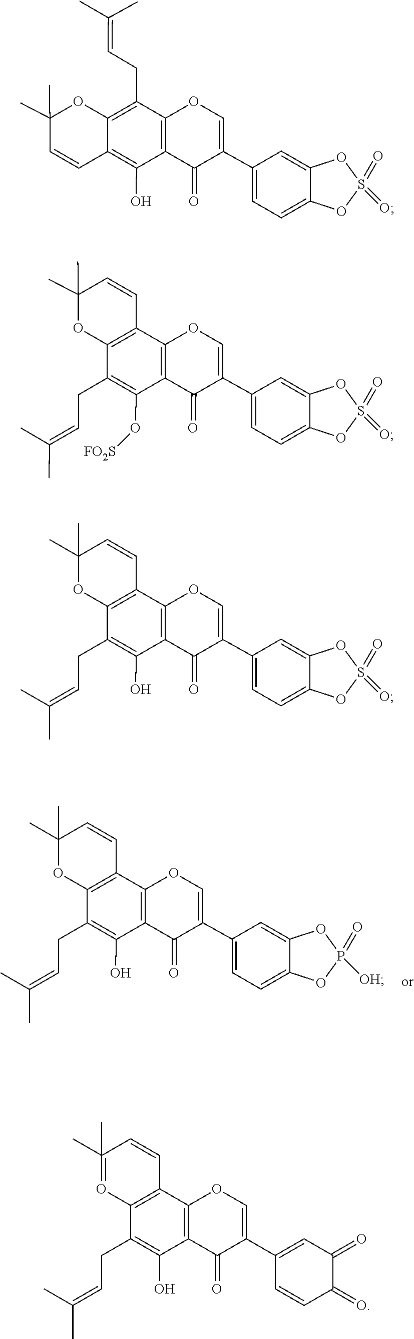

Also disclosed are embodiments of a therapeutically effective composition comprising one or more such compounds, and a pharmaceutically acceptable carrier.

The disclosed compounds may be used in suppressing or preventing tumor growth without suppressing immune cell function. Also disclosed are method embodiments of inhibiting a Crk-mediated signaling pathway, comprising exposing a sample or a subject to a compound according to any or all of the above embodiments, or a therapeutically effective composition thereof.

Also disclosed are embodiments of a method of augmenting immune cell response in a subject having, or suspected of having, a chronic viral infection or a cancer, comprising administering the subject to a compound according to any or all of the above embodiments, or a therapeutically effective composition thereof.

The foregoing and other objects and features of the present disclosure will become more apparent from the following detailed description, which proceeds with reference to the accompanying figures.

### BRIEF DESCRIPTION OF THE DRAWINGS

FIGS. 1A-1D show results from an alpha assay as described herein, wherein FIG. 1A shows a representative detection schematic using an alpha assay, which uses two beads: Nickel-coated donor beads which bind his-tagged Crk, and anti-Rabbit IgG-coated acceptor beads. Ab1 phosphorylation of CrkL (Tyr207) can be detected with an anti-pCrkL antibody (Rabbit IgG).

Each bead contains a different proprietary mixture of chemicals, key elements of the Alpha technology. Donor beads contain a photosensitizer, phthalocyanine, which converts ambient oxygen to an excited and reactive form, singlet oxygen, upon illumination at 680 nm. Within its four μsec half-lives, singlet oxygen can diffuse approximately 200 nm in solution. If an acceptor bead is within that distance, energy is transferred from the singlet oxygen to thioxene derivatives within the acceptor bead, resulting in light production at 520-620 nm. Modified from PerkinElmer;

FIG. 1B shows results for five libraries, as indicated, that were screened and plotted as a function of normalized intensity from each AlphaScreen (two experiments). Pomiferin (also referred to herein as NC5) (indicated in purple) was identified from both NCI natural product and nature compound (NC) libraries; FIG. 1C shows that the pCrkL antibody verified increased emission intensity in a dose-dependent manner; and FIG. 1D shows that there were eight promising hits (#5-12) from the NC library. Gleevec blocked the interaction between Ab1 and Crk and was used as a positive control. DMSO, vehicle control.

FIGS. 2A-2D show results for evaluating NC5 binding to CrkL, wherein FIG. 2A shows a schematic illustration of the principle of thermal shift: protein will become denatured at an increased temperature, and the denatured status can be detected by the presence of a fluorescent dye (here Sypro Orange is used). A candidate chemical that can bind to the protein will stabilize the protein and result in a higher denaturation temperature. The midpoint value of the stability curve is characterized as the melting temperature (Tₘ); FIG. 2B shows a representative demonstration of NC5 binding to CrkL, wherein the top curve (after temperature of 50° C.) represents CrkL only (without drug), and the lower curve (after temperature of 50° C.) represents CrkL+NC5; and FIGS. 2C and 2D show the statistical analysis of thermal shift for CrkL (FIG. 2C) and Ab1 (FIG. 2D), respectively. Seventy-one candidate drugs were screened using the thermal shift assay for both CrkL and Ab1. Protein only “CrkL” or “Ab1.” More than two center degrees were defined as a positive result. Each drug was screened for each protein in triplicate.

FIGS. 3A-3C show results from using an in vitro kinase assay to evaluate candidate compounds, wherein FIG. 3A shows results when Lane 1 is the marker, Lane 2 (CrkL only) represents a blank control, Lane 3 (CrkL+Ab1) represents a negative control, Lane 4 (Gleevec treatment) represents a positive control, and Lanes 5 to 13 represents CrkL+Ab1+indicated compound. An anti-CrkL antibody detected protein expression; for FIG. 3B, similar loading design as in FIG. 3A was used, but detected by anti-phosphor-CrkL antibody (pTyr 207); and FIG. 3C shows the relative kinase inhibitory efficiency based on the expression of CrkL and phosphor-CrkL by the in the vitro kinase assay. The expression of both CrkL and phosphor-CrkL in the negative control was arbitrarily set as “1”, and other treatments were relatively compared with the negative control. The pCrkL and CrkL were set as the phosphorylation ratio, while the kinase inhibition efficiency was set as (1−pCrkL/CrkL)×100%.

FIGS. 4A-4D show results from using an smFRET assay design, wherein FIGS. 4A and 4B show a schematic of the assay design, illustrating that CrkL was site-specifically labeled with donor (light lines) and acceptor (dark lines) FRET dyes. To surface immobilize, 6×His tagged CrkL was added to a biotinylated 6×His antibody that was bound to streptavidin, which was adsorbed to a quartz slide. A lipid bilayer was used to passivate the quartz surface. Phosphorylation was performed on the immobilized Crk in situ when indicated. In the absence of Ab1 kinase or the presence of pCrk inhibitor (e.g., a compound embodiment of the present disclosure), non-phosphorylated, open conformation CrkL generates low smFRET (FIG. 4A); FIG. 4B shows that, in the presence of Ab1, phosphorylated, closed conformation pCrkL generates high FRET; FIG. 4C shows examples of the donor (light lines) and acceptor (dark lines) intensity for site-specific labeled CrkL showing high FRET (upper) and low FRET (bottom); and FIG. 4D shows actual smFRET data show that phosphorylation decreases the distance between Crk's SH2 and SH3C domains. FRET is high in the presence of Ab1 (line labeled “CrkL+Ab1”) without its inhibitor-Gleevec and decreases (line labeled “CrkL+Ab1+Gleevec) with Gleevec, a positive control (upper panel), and NC5 (bottom right panel).

FIGS. 5A-5E show results for analyzing breast cancer cells as described herein, wherein FIG. 5A shows results for three breast cancer cell lines, Hs578t, MDA-MB-231(MDA-231) and MDA-MB-468 (MDA-468), which were treated with DMSO (1:4000), Gleevec (2.5 μM), and NC5 (2.5 μM) for 48 hours and proliferation of cells was measured by MTT. Both Gleevec (p=0.05) and NC5 (p=0.0009) significantly inhibited cell proliferation in Hs578T cells (upper panel); NC5 (p=0.02), but not Gleevec (p=0.27) inhibited cell proliferation in MDA-MB-231 cells (middle); NC5 (p=0.02), but not Gleevec (p=0.99) inhibited cell proliferation in MDA-MB-468 cells (lower panel); FIG. 5B shows PBMCs from three healthy donors were treated with DMSO, Gleevec, and NC5 overnight before applying to an IL2 ELISA assay. Both Gleevec and NC5 did not inhibit IL2 secretion among donor 1 (upper panel), donor 2 (middle), and donor 3 (lower panel); FIGS. 5C and 5D show that breast cancer cells are more sensitive to Pomiferin treatment than PMBC and leukemia cells, wherein in FIG. 5D, lines having the symbols ▾, ♦, ▴, and ● represent the TNBC cells (MDA-MB-231, BT549, and Hs578T), lines with the symbols ● and ▪ represent the non-TNBC cells (MCF7 and MDA-MB-453). Peripheral blood mononuclear cells (PBMCs, line with the symbol □) were used to determine the specificities of the cytotoxic effects of Pomiferin. The survival curve of PBMC-average is from four different healthy donors. Cells (1×10³−3×10³) were plated in 96-well plates, and cell growth was determined by MTT assay three days after cells were treated with Pomiferin at the indicated doses. The IC50 of Pomiferin for each cell line was calculated by The ComboSyn, Inc., the publisher of the CompuSyn software; and FIG. 5E shows results where the K562, NK92-MI, Daudi, KHYG-1, PBMC, and MDA-231-LUC cells were treated with five μM Pomiferin for three days, and cell growth was determined by MTT assay.

FIG. 6 shows structures of compounds disclosed herein, including Pomiferin (also referred to herein as NC5), compound II, and compound III. All structures of modified products were submitted for confirmation through mass spectrometry (MS).

FIGS. 7A-7C shows results using MDA-MB-231 cells (FIG. 7A) and BXPC3 cells (FIG. 7B), wherein the cells were treated with different concentrations of NC5 or compound II (referred to as “NC5-SO2F” in FIGS. 7A-7C), and viability was determined by MTT assay. Compound II had increased toxicity at 5 μM in both MDA-231 and BXPC3 cells; FIG. 7C shows the viability of NC5 or Compound II for BXPC3 cells was confirmed by PI staining.

FIGS. 8A-8C show results using MDA-MB-231 cells (FIG. 8A) or BXPC3 cells (FIG. 8B), wherein the cells were treated with 5 μM of NC5, Compound III (referred to as “NC5-140-1” in FIGS. 8A-8C), and Compound I (referred to as “NC5-140-2” in FIGS. 8A-8C) for 24-48 hours, wherein the XTT assay was used to determine cell viability. The symbol indicates the significance of NC5 vs. Compounds I and/or III; *p<0.05. Data represent mean±SEM. N=3; FIG. 8C shows that Sk-Hep1 cells were treated with 5 μM of the disclosed compound for 24 hours, the cell culture images demonstrated the growth inhibition of disclosed compounds.

FIGS. 9A-9F show results using certain compound embodiments in mice, wherein FIG. 9A shows the results using MDA-MB-231 expressing firefly luciferase (MDA-231-LUC) cells were used to generate a xenografted model of TNBC in NSG™ mice. Mice were sacrificed and dissected at the endpoint when the tumor size reached 20 mm (2.0 cm). Five mice per group were used. The line with the symbol ● represents vehicle (0.5% Avicel®); the line with the symbol ▪ represents Gleevec(imatinib mesylate, 40 μg/g) treatment; the line with the symbol ▴ represents Pomiferin (5 μg/g) treatment. The star symbols near each such lines indicate the significance of Vehicle vs. Gleevec or the significance of Gleevec vs. Pomiferin. *p<0.05, **p<0.005, ***p<0.0005 at the indicated treatment days. Statistically significant by two-way ANOVA multiple comparisons. Data represent mean±SEM. N=5; FIG. 9B shows survival of mice bearing MDA-231-LUC orthotopic xenograft tumors following treatment with vehicle (●), Gleevec (▪), or Pomiferin (▴). The P-value for a vehicle vs. Gleevec, vehicle vs. Pomiferin and Gleevec vs. Pomiferin were 0.0173, 0.0025, and 0.0025, respectively. P-values were determined by the Log-rank (Mantel-Cox) test (n=5 mice per group). The median survival for the vehicle, Gleevec, and Pomiferin were 25, 28, and 41 days, respectively; FIG. 9C shows bodyweight of the mice shows toxicity of the vehicle (●), Gleevec (▪), or Pomiferin (▴) treatment and general health of the animals. The significant difference between the Vehicle vs. Pomiferin group at day 23 shows that the tumor mass per se reached 1.5 to 2 g in the vehicle-treated group. The significant bodyweight difference between the Gleevec vs. Pomiferin group was on day 27; FIG. 9D shows four- to six-week-old female nonobese diabetic/severe combined immunodeficiency (NOD/SCID) Patient-Derived Xenografts (PDX) tumor-bearing mice with a P1-P3 fragment of a human patient-derived breast cancer xenograft TM00089 implanted subcutaneously (The Jackson Laboratory) were obtained for use in this Pomiferin therapy study. Mice were randomly divided into two groups of 5-6 mice each. Mice were dissected at the endpoint when the tumor size reached 20 mm (2.0 cm). The line with the symbol ● represents vehicle (0.5% Avicel®), and the line with the symbol u represents Pomiferin (5 μg/g) treatment. Mice were monitored, and tumor growth was documented twice weekly by caliper measurements. The P-value for a vehicle vs. Pomiferin was <0.0001. P-value was determined by the Two-way ANOVA test (n=5-6 mice per group); FIG. 9E shows survival of mice bearing PDX TM00089 tumors following treatment with vehicle (●) and Pomiferin (▪). The P-value for a vehicle vs. Pomiferin was 0.0025. P-value was determined by the Log-rank (Mantel-Cox) test (n=5-6 mice per group). The median survival for the vehicle and Pomiferin were 39 and 53 days; and FIG. 9F shows body weight of the mice indicates toxicity of the vehicle (●) and Pomiferin (▪) treatment and general health of the animals.

FIGS. 10A-10D shows results establishing that NC5 significantly inhibits tumor growth in Sk-Hep1 xenograft mice; FIG. 10A shows survival of mice SK-Hep1 xenograft tumors following NC5 treatment; FIG. 10C shows NC5 significantly reduces tumor sizes in BXPC3 xenograft mice; and FIG. 10D shows NC5 increased survival in the BXPC3 mouse model.

FIGS. 11A-11D show results for treatments with compound embodiments disclosed herein inhibits HCC and Breast tumor growth and prolongs mice survival in both xenograft mouse models; FIG. 11A shows NC5 and compound embodiments of the present disclosure (compounds III and I, referred to as “NC5-140-1” and “NC5-140-2,” respectively) inhibit tumor growth in Sk-Hep1 xenograft mice; FIG. 11B shows survival of mice SK-Hep1 xenograft tumors increased following NC5 treatment and treatment with Compounds III and I; FIG. 11C shows NC5 or compound III reduces tumor sizes in the 4T1 orthotopic breast mice model; FIG. 11D shows both NC5 and Compound III significantly increased the survival in the orthotopic breast cancer mouse model.

FIGS. 12A-12D show results for tests wherein blood samples were collected from the mice treated with vehicle Gleevec (40 μg/g), low dose of Pomiferin (5 μg/g), and high dose of Pomiferin (20 μg/g) for twenty-day treatment. Three mice were treated in each group. Blood samples were collected from every mouse in pre-treatment and post-treatment (20 days), and serum was obtained for alanine aminotransferase (ALT, SGPT, FIG. 12A), aspartate aminotransferase (AST, SGOT, FIG. 12B), blood urea nitrogen (BUN, FIG. 12C), and creatinine (FIG. 12D) tests to monitor liver and kidney toxicity. The normal Reference Ranges for ALT is 17-77 IU/L; AST is 54-298 IU/L; BUN is 18.5-33.3 mg/dL; Cr is 0-0.40 mg/dL. The results indicate that Pomiferin therapy and Gleevec at the indicated dosages result in little to no damage to the liver or kidney during treatment in mice.

FIGS. 13A-13D shows results obtained after treating the MDA-MB-231-LUC cells were treated or untreated (control) with three M Pomiferin for six days. The cells were labeled with total CrkL or phosphor-CrkL by Alexa Fluor 647 (AF-647) and stained with secondary antibodies conjugated with AF-647 as staining control. FIG. 13A shows results from flow cytometric analysis of the mean fluorescence intensity (MFI) from the fluorescence channel of AF-647 was labeled as an indicated number in each cell; FIG. 13B shows results from using Western blotting to check the basal levels of pCrkL, and CrkL in MDA-231, MDA-468 and HepG2 cells, PBMC cells from normal donors were used along as comparison. pCrkL levels in tumor cells were much higher than PBMC; FIG. 13C shows results after MDA-468 or PBMC were treated with various concentrations of NC5 for 24 hours. Cells were harvested and stained with Annexin V-FITC/PI, then measured with Flow cytometry; NC5 dramatically increased apoptosis in MDA-468 cells. But only had minimal effects in PBMC; FIG. 13D shows the apoptotic effects of NC5 in both MDA-231 and MDA-468 cells were summarized in the bar chart; even the low concentration of NC5 (1 uM) significantly induced apoptosis in TNBC. PBMC cells were used as a control and showed limited effects. The star symbol indicates the significance of vehicle vs. Pomiferin of the indicated concentration. *p<0.05, **p<0.005, ***p<0.0005. Data represent mean±SEM, n=4.

FIGS. 14A and 14B show that NC5 inhibits proliferation through the regulation of the ERK pathway in TNBC; FIG. 14A show results after cells were treated with NC5 (1 uM, 5 uM and 10 uM) for 24 hours. Cell proliferation was determined by MTT assay. NC5 significantly inhibited cell proliferation in MDA-231 and MDA-468 cells but not in PBMC. The star symbol indicates the significance of vehicle vs. Pomiferin of the indicated concentration. *p<0.05, **p<0.01, ***p<0.001. Data represent mean±SEM, n=4; FIG. 14B shows results after MDA-231, MDA-468 and PBMC cells were treated with various concentrations of NC5 for 24 hours. The protein levels of pCrkL, CrkL, cell proliferation- and apoptosis-related targets were determined by Western blotting. pCrkL levels were dramatically inhibited by NC5 treatment. β-actin was used as the loading control. The Integrated Density Values (IDV) of bands for blots are shown under each blot.

FIG. 15 is a schematic illustrating the proposed role of pCrkL in the balance of proliferation inhibition and apoptosis induction in tumor or normal cells. NC5 specifically inhibited pCrkL in TNBC, which leads to inhibition of proliferation and increase of apoptosis. These events are mainly due to the regulation of downstream target proteins indicated in the diagram. At the same time, NC5 had minimal toxicity in PBMC from healthy donors. The selective toxicity of NC5 makes it an ideal single or combinational agent for chemotherapy in different solid tumor cells, with few side effects toward normal cells.

FIG. 16 is a plot showing results obtained after using flow cytometry to analyze SK-Hep1 cells pretreated with 50 uM H₂O₂ for 1 hour to induce activation of pCrkL, followed by incubation with 10 uM NC5 or Compound III for 24 hours; pCrkL (T207) expression was determined by flow cytometry. Both NC5 and Compound III inhibited H₂O₂ induced pCrkL protein levels in Sk-Hep1 cells.

FIGS. 17A and 17B are graphs showing the effects of NC5 on PBNK (FIG. 17A) or CAR-NK (FIG. 17B) killing towards BXPC3.

FIGS. 18A and 18B are graphs showing the effects of NC5 and Compound III on PBNK killing towards MDA-MB-231 (FIG. 18A) and SK-Hep1 (FIG. 18B).

FIGS. 19A-19E are graphs of cell viability as a function of concentration showing results obtained from analyzing the activity of NC5, Compound III, and either Cytarabine (FIG. 19A) or Sorafenib (FIGS. 19B-19E) in different cell lines, including (i) the human acute monocytic leukemia (AML) cell line MoLM-13 (FIG. 19A), from the peripheral blood of a patient at relapse of AML; (ii) the HepG2 cell line (FIG. 19B); (iii) the SKHep1 cell line (FIG. 19C); (iv) the Huh7 cell line (FIG. 19D); and the MDA 468 cell line (FIG. 19E).

### DETAILED DESCRIPTION

### Overview of Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

Compounds herein can include all stereoisomers, enantiomers, diastereomers, mixtures, racemates, atropisomers, and tautomers thereof.

Compounds disclosed herein may contain one or more asymmetric elements such as stereogenic centers, chiral axes and the like, e.g., asymmetric carbon atoms, so that the chemical conjugates can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

All forms (for example solvates, optical isomers, enantiomeric forms, polymorphs, free compound and salts) of a disclosed compound may be employed either alone or in combination.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, methyl refers to both CH₃ or CH₃ wherein from 1 to 3 hydrogens are replaced by deuterium, such as in CDₓH₃₋ₓ.

To facilitate review of the various embodiments of this disclosure, the following explanations of specific terms are provided.

Administer, Administering, Administration: As used herein, administering a compound of the present disclosure, or a therapeutically effective composition thereof, to a subject means to apply, give, or bring the agent into contact with the animal, by any effective route. Administration can be accomplished by a variety of routes, such as, for example, parenterally, such as intravenous administration. In some examples, administration may be parenterally, for example intravenously; however, other routes of administration can be utilized, such as oral administration. Appropriate routes of administration can be determined based on factors such as the subject, the condition being treated, and other factors.

Cancer: Also referred to as a “malignant tumor” or “malignant neoplasm,” cancer refers to any of a number of diseases characterized by uncontrolled, abnormal proliferation of cells. Cancer cells have the potential to spread locally or through the bloodstream and lymphatic system to other parts of the body (e.g., metastasize) with any of a number of characteristic structural and/or molecular features. A “cancer cell” is a cell having specific structural properties, lacking differentiation, and being capable of invasion and metastasis. Indolent and high-grade forms are included.

Contacting or Exposing: Placement in direct physical association, including both a solid and liquid form. In one example, contacting includes association between a substance or cell in a liquid medium and one or more compounds according to the present disclosure. Contacting and/or exposing can occur in vitro with isolated cells or tissue or in vivo by administering to a subject.

Pharmaceutically acceptable carrier: Remington: The Science and Practice of Pharmacy, The University of the Sciences in Philadelphia, Editor, Lippincott, Williams, & Wilkins, Philadelphia, PA, 21^{st} Edition (2005) describes compositions and formulations suitable for pharmaceutical delivery of one or more therapeutic compositions, such as compositions comprising a compound embodiment according to the present disclosure.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Pharmaceutically Acceptable Salt: Pharmaceutically acceptable salts of compound embodiment described herein that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compound embodiments form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

Prodrug: Compound embodiments disclosed herein that are transformed, most typically in vivo, to yield a biologically active compound, particularly the parent compound, for example, by hydrolysis in the gut or enzymatic conversion. Common examples of prodrug moieties include, but are not limited to, pharmaceutically acceptable ester, carbonate, amide, and/or carbamate forms of a compound having an active form bearing a carboxylic acid moiety and/or a hydroxyl group. Examples of pharmaceutically acceptable esters of the compound embodiments of the present disclosure include, but are not limited to, esters of phosphate groups and carboxylic acids, such as aliphatic esters, particularly alkyl esters (for example C₁₋₆alkyl esters). Other prodrug moieties include phosphate esters, such as —CH₂—O—P(O)(OR^{a})₂ or a salt thereof, wherein Ra is hydrogen or aliphatic (e.g., C₁₋₆alkyl). Acceptable esters also include cycloalkyl esters and arylalkyl esters such as, but not limited to, benzyl. Examples of pharmaceutically acceptable amides of the compound embodiments of this disclosure include, but are not limited to, primary amides, and secondary and tertiary alkyl amides (for example with between one and six carbons). Amides and esters of disclosed exemplary embodiments of compound embodiments according to the present disclosure can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

Subject: A living multi-cellular vertebrate organism, a category that includes both human and non-human mammals (such as veterinary animals, including dogs and cats, as well as mice, rats, rabbits, sheep, horses, cows, and non-human primates).

Treating or inhibiting a disorder: “Inhibiting” a disease or disorder refers to inhibiting the full development of a disease or disorder, for example, a cancer (e.g., a tumor or hematological malignancy). Inhibition of a disease or disorder can span the spectrum from partial inhibition to substantially complete inhibition (e.g., including, but not limited to prevention) of a disease or disorder (such as a cancer). In some examples, the term “inhibiting” refers to reducing or delaying the onset or progression of a disease or disorder. “Treatment” refers to a therapeutic intervention that ameliorates a sign or symptom of a disease or disorder after it has begun to develop. The term “ameliorating,” with reference to a disease or disorder, refers to any observable beneficial effect of the treatment. The beneficial effect can be evidenced, for example, by a delayed onset of clinical symptoms of the disease or disorder in a susceptible subject, a reduction in severity of some or all clinical symptoms of the disease or disorder, a slower progression of the disease or disorder, an improvement in the overall health or well-being of the subject, or by other parameters well known in the art that are specific to the particular disease or disorder, such as improved survival of a subject having a cancer. Treatment may be assessed by objective or subjective parameters; including, but not limited to, the results of a physical examination, imaging, or a blood test. A “prophylactic” treatment is a treatment administered to a subject who does not exhibit signs of a disease or disorder or exhibits only early signs for the purpose of decreasing the risk of developing pathology, such as to prevent the occurrence or recurrence of a cancer. A subject to be administered an effective amount of the disclosed compound embodiments can be identified by standard diagnosing techniques for such a disease or disorder, for example, presence of the disease or disorder or risk factors to develop the disease or disorder.

Therapeutically Effective Amount: An amount of a compound embodiment sufficient to inhibit a CrkL and/or treat a specified disorder or disease, or to ameliorate or eradicate one or more of its symptoms and/or to prevent the occurrence of the disease or disorder. The amount of a compound which constitutes a “therapeutically effective amount” will vary depending on the compound, the disease state and its severity, the age of the patient to be treated, and the like. The therapeutically effective amount can be determined by a person skilled in the art with the benefit of the present disclosure.

### Introduction

The Crk family proteins, including CrkI, CrkII, and CrkL, belong to an adaptor protein family, originally discovered from the oncogene fusion product of the chicken tumor no. 10 (CT10) retrovirus.

The CrkL and CrkII proteins contain one Src homology-2 (SH2) domain and two SH3 domains and lack intrinsic enzymatic activity. Phosphorylation of Crk proteins is one of the most important regulators of their functions. Specifically, a tyrosine site (Y207) in the linker region of CrkL between the two SH3 domains can be phosphorylated by kinases (such as BCR-ABL), leading to the formation of an intramolecular interaction in which phosphorylated Y207 binds to its SH2 domain. The intramolecular interaction decreases the possibility that the pCrkL SH2 domain will bind to other molecules. Therefore, phosphorylated CrkL usually functions as a negative regulator.

Several groups report that Crk family proteins are upregulated in various cancers. Specifically, Crk family proteins are upregulated in tumor cell lines and tumor tissues compared to their corresponding benign tissues or adjacent normal tissues, such as in breast cancer, lung carcinomas, hematopoietic cancers, glioblastoma, ovarian cancer, colon cancer, sarcomas, and melanoma. Interestingly, a high level of CrkL can be used as a novel prognostic marker in hepatocellular carcinoma (HCC).

Given that elevated expression of Crk family proteins and increased pCrk are seen in various cancers (e.g., lung, breast, and ovarian), the disclosed pCrk inhibitors are likely to have a broad clinical impact.

Crk family proteins play not only important roles in cancer but also regulate immune responses. Specifically, CrkL interacts with hematopoietic progenitor kinase 1 (HPK1) to affect interleukin-2 (IL-2) secretion in T cells. CrkII forms a complex with Cb1 to affect B-cell antigen receptor (BCR) signaling, but without phosphorylation of CrkII. Crk plays a role in NK cells inhibitory receptor signaling and modulates the signaling of activating receptors.

As there are numerous ways in which Crk family proteins are implicated in cancer and immune responses, blocking phosphorylation of Crk or otherwise inhibiting Crk functions in tumor and exhausted immune cells (e.g., PD-1 upregulated CTL or NK cells) should not only inhibit tumor progression but also restore effective immune responses against tumors. An AlphaScreen to identify inhibitors of phosphorylated Crk or Crk protein function, focusing on the CrkL protein in the current study. 71 candidate compounds from five libraries (including more than 2000 compounds) were identified using the AlphaScreen as an initial screening strategy. One identified hit was Pomiferin.

Therapeutic activity of the disclosed compounds is evaluated in several in vivo tumor cancer mouse xenograft models. In particular embodiments, the disclosed compounds can inhibit tumor cell growth by inhibiting pCrk functions. The mechanistic study found that cell growth inhibition was mainly due to increased apoptosis induced by disclosed compound embodiments. The inhibition of pCrkL also increased the protein levels of pJNKs, and subsequentially increased the expression of pro-apoptotic protein BIM in tumor cells, but not PBMC. Therefore, the therapeutic use of a small molecule inhibitor of pCrk for cancer treatment is feasible.

### Compounds

Disclosed herein are compound embodiments that are CrkL inhibitors. In particular embodiments, the compounds are selected from the compounds illustrated below, as well as any pharmaceutically acceptable salts, prodrugs, isomers, or solvates thereof. In an independent embodiment, the compound is not, or is other than, Pomiferin (also known as “3′,4′,5-Trihydroxy-6″,6″-dimethyl-6-(3-methylbut-2-en-1-yl)-6″H-pyrano[2″,3″:7,8]isoflavone” or “3-(3,4-Dihydroxyphenyl)-5-hydroxy-8,8-dimethyl-6-(3-methylbut-2-en-1-yl)-4H,8H-(benzo[1,2-b:3,4-b′]dipyran)-4-one”).

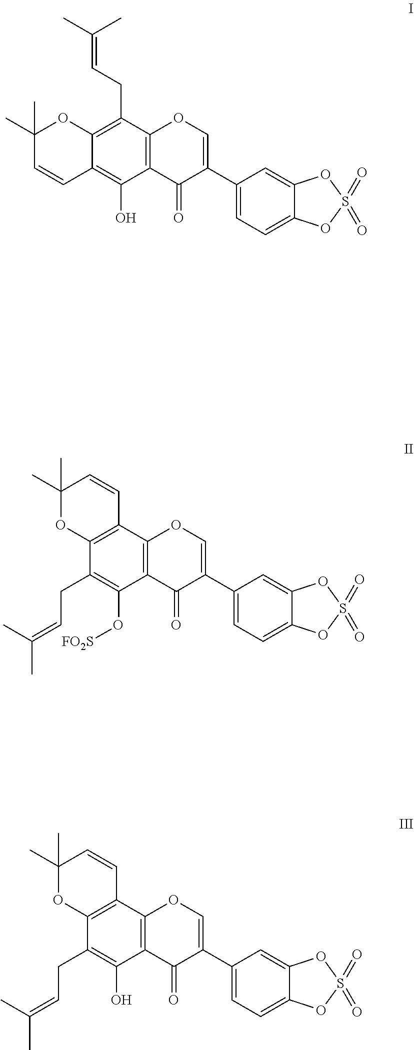

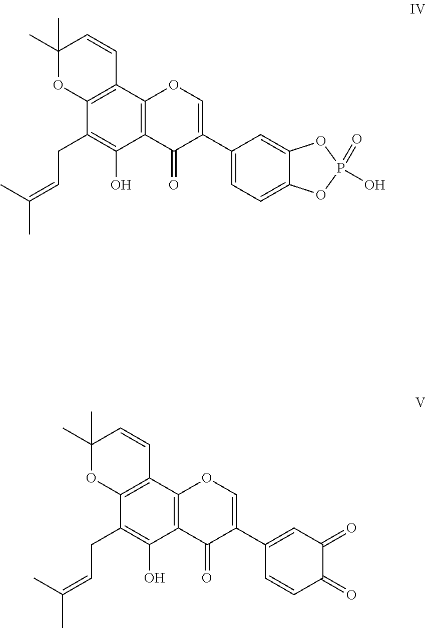 [SMILES: CC(C)=CCc1c2c(c3occ(-c4ccc5c(c4)OP(=O)(O)O5)c(=O)c3c1O)C=CC(C)(C)O2 ; CC(C)=CCc1c2c(c3occ(C4=CC(=O)C(=O)C=C4)c(=O)c3c1O)C=CC(C)(C)O2]

Also disclosed are embodiments of a therapeutically effective composition that can comprise one or more of the compound embodiments illustrated below. Such composition embodiments can further comprise a pharmaceutically acceptable carrier. The disclosed therapeutically effective composition embodiments typically comprise the compound in a therapeutically effective amount. In some additional embodiments, the amount of the compound ranges from greater than 0% up to 99% total weight percent, such as from greater than 0 wt % to 95 wt %, such as greater than 0 wt % to 90% wt %, or greater than 0 wt % to 85 wt %, or greater than 0 wt % to 80 wt %, or greater than 0 wt % to 75 wt %, or greater than 0 wt % to 70 wt %, or greater than 0 wt % to 65 wt %, or greater than 0 wt % to 60 wt %, or greater than 0 wt % to 55 wt %, or greater than 0 wt % to 50 wt % based on the total weight percent of the therapeutically effective composition. In some embodiments, therapeutically effective composition a compound disclosed herein can comprise from greater than 0 wt % to 95 wt %, such as 0.001 wt % to 95% wt %, or 0.01 wt % to 95 wt %, or 0.1 wt % to 95 wt %, or 1 wt % to 95 wt %, or 5 wt % to 95 wt %, or 10 wt % to 95 wt %, or 25 wt % to 95 wt % of the compound based on the total weight percent of the therapeutically effective composition. In some embodiments, any remaining weight percent of the composition can be made up by a pharmaceutically acceptable carrier.

The compounds disclosed herein (or any therapeutically effective compositions thereof) can be administered in the form of solids, liquids, and/or lotions. Suitable solid forms of administration include, but are not limited to, tablets, capsules, powders, solid dispersions, and the like containing the compounds disclosed herein (or any therapeutically effective compositions thereof). Suitable liquid or lotion forms include, but are not limited to, oil-in-water or water-in-oil emulsions, aqueous gel compositions, or liquids or lotions comprising the compounds disclosed herein (or any therapeutically effective compositions thereof) formulated for use as foams, films, sprays, ointments, pessary forms, suppository forms, creams, liposomes or in other forms embedded in a matrix for the slow or controlled release of the compounds disclosed herein (or any therapeutically effective compositions thereof) to the skin or surface onto which it has been applied or is in contact. In particular disclosed embodiments, a dermal patch can be used to facilitate dosing and delivering the compounds disclosed herein (or any therapeutically effective compositions thereof). In yet additional embodiments, the compounds disclosed herein (or any therapeutically effective compositions thereof) can be formulated as a gel for topical administration.

The compounds disclosed herein (or any therapeutically effective compositions thereof) may be formulated so as to be suitable for a variety of modes of administration, including, but not limited to, topical, ocular, oral, buccal, systemic, nasal, injection (such as intravenous, intraperitoneal, subcutaneous, intramuscular, or intrathecal), transdermal (e.g., by mixing with a penetrating agent, such as DMSO), rectal, vaginal, a form suitable for administration by inhalation or insufflation, a form suitable for implantation, or any combination thereof.

For oral or buccal administration, the compounds disclosed herein (or any therapeutically effective compositions thereof) may take the form of lozenges, tablets, or capsules prepared by using conventional means with pharmaceutically acceptable excipients that would be recognized by people of skill in the art with the benefit of the present disclosure. The tablets or capsules may be coated by methods well known in the art with, for example, sugars, films, or delayed-release, sustained-release, and/or enteric coatings.

Liquid preparations of the compounds disclosed herein (or any therapeutically effective compositions thereof) for oral administration may take the form of, for example, elixirs, solutions, syrups or suspensions, or they may be presented as a dry product for constitution with water or other suitable vehicle before use. Preparations for oral administration also may be suitably formulated to give controlled release of the compound or the composition.

For topical administration, the compounds disclosed herein (or any therapeutically effective compositions thereof) can be formulated as solutions, lotions, gels, ointments, creams, suspensions, and the like. For transmucosal administration, penetrants appropriate to the barrier to be permeated can be used.

Systemic formulations include those designed for administration by injection, e.g., subcutaneous, intravenous, intramuscular, intrathecal or intraperitoneal injection, as well as those designed for transdermal, transmucosal oral or pulmonary administration. Useful injectable preparations include sterile suspensions, solutions or emulsions of the compounds disclosed herein (or any therapeutically effective compositions thereof) in aqueous or oily vehicles. The composition may also contain formulating agents, such as suspending, stabilizing and/or dispersing agents.

For rectal routes of administration, the compounds disclosed herein (or any therapeutically effective compositions thereof) may be formulated as solutions (for retention enemas) suppositories or ointments containing conventional suppository bases, such as cocoa butter or other glycerides.

For nasal administration or administration by inhalation or insufflation, the compounds disclosed herein (or any therapeutically effective compositions thereof) can be conveniently delivered in the form of an aerosol spray from pressurized packs or a nebulizer with the use of a suitable propellant. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compounds disclosed herein (or any therapeutically effective compositions thereof) and a suitable powder base such as lactose or starch.

In some embodiments, compound IV above can be made by treating Pomiferin with an oxo-rhenium(VII) catalyst to promote a dehydrative condensation reaction between phosphoric acid and the 1,2-diol groups of Pomiferin. In yet other embodiments, bis(dimethylamino)phosphorodiamidate can be used to promote this conversion to arrive at compound IV. In some embodiments, compound V can be made by exposing Pomiferin to a suitable oxidant so as to oxidize the 1,2-diol groups to the corresponding oxo groups, such as is shown in compound V. In some embodiments, compound II can be made by exposing Pomiferin to a base (e.g., Et₃N) and sulfuryl fluoride (SO₂F₂) or by exposing Pomiferin to 4-[(acetylamino)phenyl]imidodisulfuryl difluoride reagent (AISF) in the presence of 1,8-diazabicyclo[5.4.0]undec-7-ene (DBU) in tetrahydrofuran (THF). In yet additional embodiments, the 1,2-diol of compounds I-III can be protected by reacting Pomiferin or the sulfonyl fluoride intermediate with SO₂F₂, as indicated in Scheme 1 below.

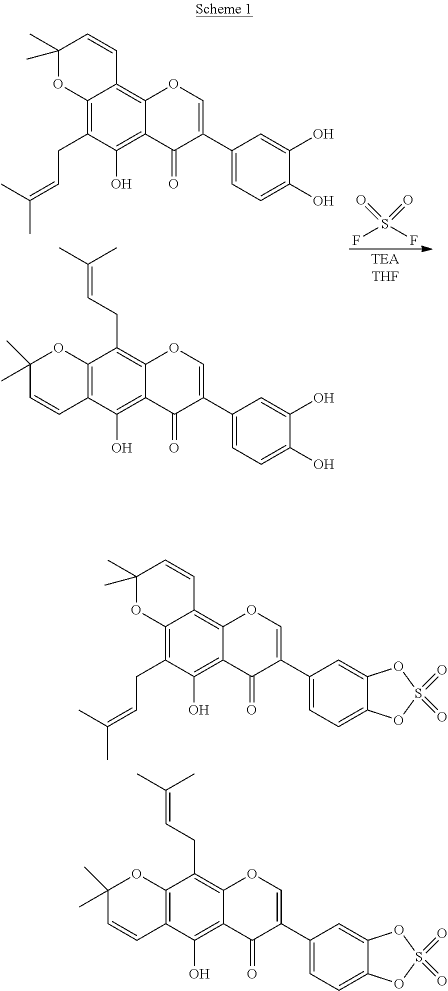

### METHODS

Disclosed herein is a new therapeutic strategy that not only can inhibit cancer cell proliferation directly but also can restore exhausted immune cell functions can be developed by targeting pCrkL, a common, master signaling molecule triggered by a variety of inhibitory receptors such as NKG2A and PD-1. In particular embodiments, a method of using a compound according to the present disclosure is described, wherein the method comprises exposing a sample or a subject to a compound of the disclosure, or a therapeutically effective composition thereof. In some embodiments, the method is a method of inhibiting a Crk-mediated signaling pathway or a method of augmenting immune cell response in a subject having, or suspected of having, a chronic viral infection or a cancer. In particular embodiments, the compound is one of Compounds I-V disclosed herein, with representative embodiments using Compound III. In particular embodiments, Compound III shows better performance in method embodiments disclosed herein as compared with Pomiferin.

Phosphorylated CrkL (pCrkL) inhibitors can be used to augment immune cell responses in chronic viral infections and cancers. Data demonstrating phosphorylation-driven conformational changes in CrkL are disclosed herein along with pCrkL inhibitor compounds, which are disclosed herein. These newly identified pCrkL inhibitors have the potential to dramatically alter the face of cancer therapy and treatment for infectious diseases (e.g., chronic HIV infection) by preventing downregulation of immune responses to cancer and infectious diseases while simultaneously directly inhibiting cancer cells or infected cells. Unlike efforts to boost T cell responses by targeting individual inhibitory receptors (e.g., PD-1, CTLA-4, LAG-3, and CD160) using antibodies, the proposed strategies are the first to address CrkL, the common downstream, central signaling hub for several inhibitory receptors in immune cells and cell proliferation in cancer cells. In addition, as increased inhibitory receptor expression accompanies other chronic viral infections, and elevated expression and increased phosphorylation of Crk family proteins are seen in a variety of cancers (e.g., lung, breast, and ovarian), pCrk inhibitors are likely to have a broad clinical impact.

Although SHP-2 is one of the well-recognized downstream signaling molecules of PD-1 signaling, no specific SHP-2 inhibitor is available, clinically, despite a three-decade-long effort. Data provided herein show that Crk functions as a molecular switch for controlling cytotoxic lymphocyte activation by switching between phosphorylated and non-phosphorylated states. Phosphorylation of Crk can be induced by different inhibitory receptors such as CD94/NKG2A, KIR2DL1 and KIR2DL2 (manuscript in preparation), and PD-1 (manuscript in preparation) in different types of cytotoxic lymphocytes (including NK cells and CTLs). Thus, pCrk is an effective therapeutic target.

Both proprietary and public, existing chemical libraries contain thousands of chemical compounds, many of which have been put to excellent therapeutic use. Gleevec, an inhibitor of Ab1 kinase used for the treatment of leukemia, is one such example. Gleevec, however, cannot be used to restore exhausted immune cells, as Ab1, in addition to phosphorylating Crk, is required for T cell activation. In addition, typical drug screening methods for small-molecule inhibitors are expensive and time-consuming, and some, like ³²P phosphor-transfer assays, have obvious safety implications. Herein, a unique combination of high-throughput AlphaScreen, kinase and thermal shift assays, and smFRET techniques are used to identify and characterize numerous pCrkL inhibitors. Using pCrk inhibitors to restore exhausted immune cell function, thus controlling tumor growth and infection, represents a conceptual and technical innovation in cancer immunotherapy by restoring the functions of exhausted immune cells and inhibiting tumor growth simultaneously.

Recently, CrkL is emerging as an important player in both cancer and immune responses. The phosphorylation status of CrkL is highly regulated in different conditions. In cancers such as acute lymphoblastic leukemia, phosphorylated CrkL or CrkII can be used as a biomarker to monitor the assessment of BCR-ABL activity in patients. In the human immune system, such as in NK cells, CrkL functions as a molecular switch based upon its phosphorylation status. CrkL has oncogenic potential and is over-expressed in numerous cancers, which is correlated with cancer malignancy status. On the other hand, CrkL also plays a very important role in immune responses.

As such important roles of CrkL have been documented in both human cancers and the immune system, drugs that can specifically block CrkL function are needed in the art. Theoretically, inhibition of phosphorylated CrkL results in dysfunctional cancer cells and enhances immune cell functions. Based on this knowledge, a potential drug to inhibit CrkL phosphorylation will be bi-functional. Unfortunately, no drug was reported to inhibit CrkL or phosphorylated CrkL in previous studies aside from siRNAs or miRNAs to target CrkL expression at the transcriptional level. As disclosed herein, an AlphaScreen technique is used to discover new drugs based on the in vitro kinase activity of Ab1 on CrkL proteins. The Alpha signal can detect this kind of kinase activity, which can also be blocked by drug-mediated interruption of the Ab1 and CrkL interaction. Five libraries (2312 drugs in total) were screened, and 71 drugs were found positive in the results. A thermal shift was also conducted to confirm the interaction between proteins (Ab1 or CrkL) and candidate drugs, finding five drugs that bind to Ab1 and five that bind to CrkL. Then, the effect of candidate drugs on CrkL phosphorylation was confirmed with two methods: in vitro kinase assay and single molecular FRET. After in vitro confirmation, functional assays were conducted in both cancer cells and immune cells. Pomiferin (also referred to herein as “NC5”) inhibits breast cancer cell proliferation but does not inhibit immune cell function. Pomiferin can efficiently repress TNBC tumor growth in two different xenograft animal models established using the MDA-MB-231 cell line and the TNBC patient tumor. No physiological appearance change, liver or kidney toxicity was detected during Pomiferin treatment, thus indicating preclinical safety of the drug. These data suggest that Pomiferin therapy could be a promising anti-cancer drug alone or clinically combined with other drugs.

It has been reported that overexpression of CrkL occurs in various tumor cells. Although the importance of CrkL and phosphorylated CrkL was reported from discovering this oncogenic protein, there are no known specific drugs that can inhibit its function. In a previous study, CrkL was found to be phosphorylated by BCR-ABL in chronic myelogenous leukemia, and phosphorylated-CrkL was found to function as a monitor for ABL kinase activity. Therefore, drugs were screened to target Ab1 activity in most studies. Few studies focused on how to inhibit CrkL phosphorylation. Although Gleevec is an excellent inhibitor of Ab1 kinase, which directly phosphorylates Crk proteins, Gleevec cannot be used to restore exhausted immune cell functions, as Ab1, in addition to phosphorylating Crk, is required for T-cell activation. This study identified a specific CrkL inhibitor, which can be used as a potential treatment for other cancers.

Candidate compounds that not only inhibited cancer cell function but also restored the exhausted immune cell function based on CrkL's roles in both cancer and immune cells were screened. The highly specific Pomiferin inhibited several solid tumor cell proliferation, such as breast cancer, HCC and PDAC cells, but did not affect immune cell functions. Pomiferin also specifically influenced several CrkL associated signaling pathways, which regulated the cell proliferation and apoptosis in TNBC and HCC, but limited effects in the immune cells. Therefore, one advantage of Pomiferin is that it does suppress solid cancer cell growth in vivo without affecting immune cell functions. Compound embodiments disclosed herein, including Compounds I-V exhibit superior inhibitory effects relative to Pomiferin. As such, these compounds can be used in method embodiments of the present disclosure.

### OVERVIEW OF SEVERAL EMBODIMENTS

Disclosed herein are compounds selected from

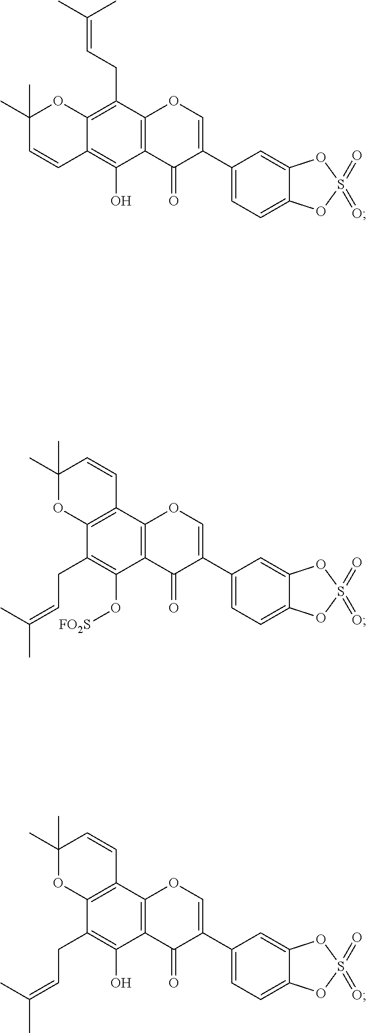

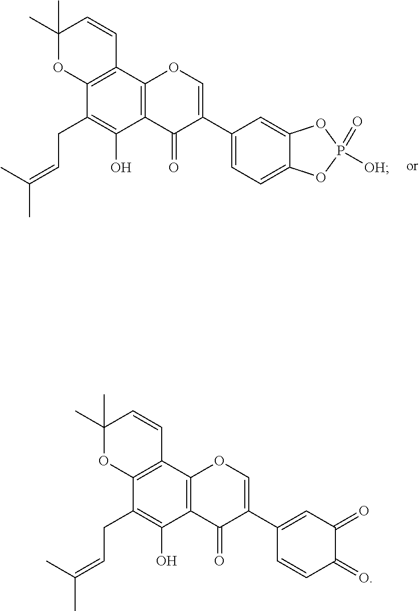 [SMILES: CC(C)=CCc1c2c(c3occ(-c4ccc5c(c4)OP(=O)(O)O5)c(=O)c3c1O)C=CC(C)(C)O2 ; "or" ; CC(C)=CCc1c2c(c3occ(C4=CC(=O)C(=O)C=C4)c(=O)c3c1O)C=CC(C)(C)O2]

Also disclosed herein are embodiments of a therapeutically effective composition, comprising a compound according to any of the above compounds and a pharmaceutically acceptable carrier.

In any or all of the above embodiments, the compound is formulated for use in suppressing or preventing tumor cell growth without suppressing immune cell function.

Also disclosed herein are embodiments of a method of inhibiting a Crk-mediated signaling pathway, comprising exposing a sample or a subject to a compound according to any or all of the above embodiments, or a therapeutically effective composition thereof.

In any or all of the above embodiments, the sample is an in vitro or ex vivo sample.

In any or all of the above embodiments, the compound inhibits tumor cell line proliferation and does not suppress immune cell function.

Also disclosed herein are embodiments of a method of augmenting immune cell response in a subject having, or suspected of having, a chronic viral infection or a cancer, comprising administering the subject to a compound according to any or all of the above embodiments, or a therapeutically effective composition thereof.

### . EXAMPLES

### Example 1

In this example, Compounds III and I as described herein were synthesized. To a solution of a Pomiferin and Aurisculasin mixture (1:1) (250 mg, 0.59 mmol) in THF (25 mL), TEA (0.41 mL, 2.97 mmol, 5 eq) was added. After cooling at −78° C., a stream of sulfuryl difluoride was bubbled through the solution for 20 seconds. The reaction was allowed to warm to room temperature and stirred over two days. The solvent was removed under reduced pressure. FCC (20% EtOAc/Hex) provided two samples: Compound III as a yellow solid (107 mg); and Compound I as a colorless foam (98 mg).

### Example 2

Drugs and source of compound libraries—NC5 (also known as Pomiferin) was purchased from MicroSource Discovery systems, Inc (Gaylordsville, CT). Five chemical compound libraries of potential inhibitors of phosphorylation of CrkL at Y207 were obtained from several sources, including Enzo Screen-Well Kinase Inhibitors (# of compounds=80), Enzo Screen-Well Phosphatase Inhibitors (# of compounds=33), MicroSource Natural Products (# of compounds=800), NCI Natural Product Set II (# of compounds=119), and Sigma LOPAC (# of compounds=1280). The majority of the compound libraries were provided by the Center for Drug Discovery in Baylor College of Medicine. Parts of compound libraries were purchased from MicroSource Discovery Inc.

Cell Culture—Human Peripheral Blood Mononuclear Cells (PBMCs) isolated from healthy donors' peripheral blood were cultured in R10 medium (RPMI medium 1640, Corning™) 10% fetal bovine serum (FBS), 100 U/mL Penicillin-Streptomycin (Corning™), two mM L-glutamine (Corning™), and 20 mM HEPES (Sigma) and adjusted to a pH of 7.0. The breast cancer cell lines were cultured in Gibco™ Dulbecco's Modified Eagle Medium: Nutrient Mixture F-12 (10% FBS and 100 U/mL Penicillin-Streptomycin). HCC cell lines used in this example, including HepG2 and Sk-Hep1 cells, were cultured in DMEM with 10% FBS. The PDAC cell line, such as BXPC3 cells, were cultured with an R10 medium.

Antibodies and Reagents—For the AlphaScreen assay, both nickel chelate donor beads attached to 6×Histidine-tagged CrkL and anti-rabbit IgG acceptor beads attached to Tyr207 antibody were purchased from PerkinElmer. Antibodies for Western blots were purchased from Cell Signaling Technologies (Danvers, MA) include the following: pCrkL (Tyr207, #3181), pErkl/2 (Thr202/Tyr2O4, #4370), pJNK1/2 (Thr183/Tyr185, #9255), ERK1/2 (#9102), JNK1/2 (#9252), Bim (#2819), and HRP-linked anti-rabbit (#7074). CrkL antibody (C-20) was purchased from Santa Cruz Biotechnology. The β-actin antibody was obtained from Millipore-Sigma (St. Louis, MO). For the thermal shift assay, SYPRO® Orange Protein Gel Stain was purchased from Life technology. For the proliferation assay, the MTT (3-[4,5-dimethylthiazol-2-yl]-2,5 diphenyltetrazolium bromide) cell proliferation assay kit was purchased from Cayman Chemical. For the IL-2 ELISA assay, the human IL-2 ELISA kit was purchased from Ray Biotech.

Cell Transfection and Infection—Initially, CrkL cDNA was ligated into the overexpression plasmid vector pCMV6-XL4, purchased from OriGene Technologies (Rockville, MD, USA). Then, HEK 293T and K562 cells were transfected with CrkL (and GFP control plasmid) plasmids using Amaxa Nucleofection technology as per the manufacturer's protocol. Briefly, both HEK 293T and K562 cells were transfected with Solution V. Cells and were mixed with plasmid DNA in Nucleofector Solution V. The cells were then transferred into cuvettes and used the appropriate Nucleofector program. For example, the Q-001 and T-016 programs were used for HEK 293T and K562 cell transfection, respectively.

Amplified Luminescent Proximity Homogeneous Assay Screen (AlphaScreen)—The AlphaScreen assay was developed to detect conformational changes due to protein-protein interactions as well as protein phosphorylation. This assay was used to screen compounds from each of the five libraries mentioned above to identify those that would most effectively inhibit phosphorylation of CrkL at Y207. Briefly, 0.5 μg/ml Ab1 was mixed in the presence of 0.5 μg/ml CrkL, 10 μm ATP and 0.2 mg/ml pCrkL in a 15-μl volume. All the reagents were diluted in kinase buffer (1 mM DTT, 0.2% w/v BSA, 100 mM Nalco, 0.01% w/v Tween-20, 1.0 mM MgC12, 20 mM Tris, adjusted to pH 7.4). Then, 0.1 μl of 10 mg/ml Gleevec was added (imatinib mesylate, also used as a control at the same concentration as tested compounds) and incubated at Room Temperature (RT) for 2 hours. After incubation, 5.0 μl of 20 μg/ml acceptor beads ligated to pTyr207 antibody were added and incubated at RT for 1 hour. After incubation with the acceptor beads, 5.0 μl of 20 μg/ml nickel chelate donor beads attached to 6×Histidine-tagged CrkL were added and incubated at RT for 1 hour. Signaling of AlphaScreen assay was measured by Infinite M1000 Pro Plate Reader (Tecan, Switzerland).

Thermal Shift Assay—Thermal shift assay is a thermal-denaturation assay, which can measure the thermal stability of a tested protein. When a protein of interest is bound to a ligand (or drug), its melting temperature increases. For each single well assay, CrkL (1 μg per well), SYPRO® Orange (1:500 dilution from original stock), candidate drugs (same final concentration as in AlphaScreen) and reaction buffer (1×TBS) were mixed. The total volume for a single well was 10 ul. The assay was quantified by a melt curve on a real-time PCR machine, using melting temperatures from 20° C. to 95° C.

in vitro Kinase Assay—An in vitro kinase assay was conducted to detect CrkL phosphorylation by Ab1 and to determine whether candidate drugs identified in the AlphaScreen assays could inhibit phosphorylation. In a 20 μl reaction system, the following was mixed: 2.5 μg CrkL protein, 0.04 μg Ab1 protein, 40 μM of candidate drugs (the same concentration as in AlphaScreen assay) and added kinase buffer (1 mM DTT, 0.2% w/v BSA, 100 mM Nalco, 0.01% w/v Tween-20, 1.0 mM MgCl₂, 20 mM Tris, adjusted to pH 7.4) with ATP (Cell Signaling Technology, MA, USA) to 20 μl. Two compare the specific in vitro kinase activity, two different reactions (one with and one without Ab1 protein) were prepared. After mixing the kinase buffer described above (in the absence of Ab1) at 30° C. for 30 minutes, Ab1 kinase was added and incubated it at 30° C. for 1 hour. Western blot loading buffer and DTT were added to all samples and denatured them in 100° C. water for 30 minutes. A C20 antibody was used to detect the total CrkL expression, which was used as an internal control in this assay. The pTyr207 antibody detected the level of phosphorylated CrkL. Gleevec was used as a positive control in this kinase assay.

Single-molecule Fluorescence Resonance Energy Transfer (smFRET)—Single-molecule Fluorescence Resonance Energy Transfer (smFRET) was applied to monitor conformations of single molecules. CrkL has two cysteines (a relatively rare amino acid), one in its SH2 and another in SH3C domains. CrkL was mixed with a mixture of equal amounts of Alexa555-maleimide and Alexa647-maleimide (Invitrogen, USA) (10× over protein concentration) to randomly label the two cysteines. The labeled protein was purified from an unreacted dye by gel filtration. Molecules with exactly one donor and acceptor are identified in single-molecule data analysis and used for further analysis. Labeling efficiencies were 50-100% as assessed by UV-VIS absorbance spectroscopy. The FRET change between non-phosphorylated CrkL and phosphorylated CrkL was measured. The dye-labeled protein was used in in vitro kinase assays with different drugs treatments as described above. smFRET measurements were performed with a prism-type total internal refection microscope (TIRF) where FRET efficiency is calculated from measured donor and acceptor intensities (I_{d} and Iₐ, respectively) by FRET=Iₐ/(I_{d}+Iₐ), as described previously. To surface immobilize 6×His tagged CrkL, the protein was added to the biotinylated 6×His antibody (Thermo Scientific Cat #: MA1-80087) bound to streptavidin, which was adsorbed to a quartz slide. A lipid bilayer was used to passivate the quartz surface. Phosphorylation was performed on the immobilized Crk in situ.

MTT assay—The MTT assay was used to detect cell proliferation. Briefly, cells were plated in a 96-well plate at a density of 50,000 cells/well in 200 μl of culture medium with candidate drugs to be tested (DMSO as a negative control). The treated cells were incubated for four hours a 37° C. with a 5% CO₂ incubator to allow dark crystals to form at the bottom of the wells. After culturing for 48 hours, 10 ml of MTT reagent was added to each well and mixed gently for one minute. After aspirating the culture medium from each well, 100 μl of crystal dissolving solution was added to each well and incubated for 10 minutes at RT. The signal was quantified by measuring the absorbance in each sample at 570 nm using a microplate reader.

IL-2 ELISA—IL-2 in vitro enzyme-linked immunosorbent assay (ELISA) was used to measure human IL-2 productions. This assay was conducted per the user manual from the kit. In brief, human PBMCs were cultured in R10 medium with 0.5 μg/ml PHA. Then, 100 μl of the standard and sample was added to each well and incubated at 4° C. overnight. A prepared biotin antibody (100 μl) was added to each well and incubated for 1 hour at room temperature. Then, 100 μl prepared streptavidin solution was added and incubated for 45 minutes at room temperature. TMB one-step substrate reagent (100 μl) was then added to each well and incubated for 30 minutes at room temperature. Stop solution (50 μl) was then added to each well, and the results were quantified at 450 nm by Infinite M1000 Pro Plate Reader (Tecan, Switzerland) immediately.

Annexin V/PI Staining—Experimental cells were collected, washed with 1×PBS, resuspended in the binding buffer, and stained with an Annexin V-FITC assay Kit (Millipore Sigma). The cells were incubated with 50 μg/ml Annexin V in 1× binding buffer at room temperature for 15 minutes, then 10 μg/ml propidium iodide for another 15 minutes and analyzed by flow cytometry (EPICS XL). Annexin V-positive/PI-negative cells were considered early apoptotic, cells with both Annexin V and PI-positive to be late apoptotic, and Annexin V negative but PI-positive to be “necrotic.” The percent of apoptosis was calculated as the sum of the percentages of both early and late apoptotic cells.

Western blotting—According to the company's protocol (Thermo Scientific, Waltham, MA). Western blotting was performed using 20 μg of total cell extracts. Briefly, transferred membranes were incubated with primary antibodies for 1 hour overnight according to the sensitivity of each antibody, followed by HRP-linked secondary antibody for 1 hour. Each membrane was stripped and re-probed for b-actin, which was used as an internal loading control. The protein bands were visualized using a chemiluminescence detection system (Thermo Scientific). The relative optical densities of each band were quantitated and indicated numerically below the bands in each Western blot.

Xenograft studies—In this evaluation, both breast cancer cell line-derived xenograft models and patient-derived xenografts (PDX) and were used. Mouse xenograft experimental protocols were approved by the Institutional Animal Care and Use Committee (IACUC). Animals were maintained and evaluated under pathogen-free conditions following IACUC guidelines.

To establish a breast cancer cell line xenograft model, female NSG mice (8-week-old) were used. Specifically, these NSG mice were injected subcutaneously with 1×10⁶ MDA-MB-231-GFP-LUC tumor cells. Briefly, MDA-MB-231-GFP-LUC tumor cells were cultured, dissociated with 0.25% trypsin to produce a single-cell suspension, and mixed with Matrigel™ (Coning™). For each mouse, 1×10⁶ tumor cells were injected into the 4^{th} mammary fat pad.

For HCC or PDAC xenograft models, NSG mice 8-week-old) were injected subcutaneously with 1×10⁶ HepG2 or BXPC3 tumor cells mixed with Matrigel.

Syngeneic orthotopic 4T1 breast tumor-bearing mouse model was established by inoculating 5×10⁴ 4T1-Luc cells mixed with Matrigel were into the left inguinal fat pad of Balb/c mice from Jackson Laboratories. This orthotopic syngeneic breast tumor model is a tool for preclinical testing of anti-cancer drugs or immunotherapy that require an intact immune system to elicit activity.

For the patient-derived xenograft (PDX) model, a cohort of mice engrafted with the TNBC tumor model #TM00089-Breast-Tumor Markers: TNBC ER−(estrogen receptor-negative)/PR—(progesterone receptor-negative)/HER2—(human epidermal growth factor receptor two negative), BRCA1 V757fs (4 to 6-weeks-old females) were obtained from Jackson Laboratories (Bar Harbor, ME). PDX models were generated by surgically engrafting tumor cells into the mammary fat pad of NSG™ (nonobese diabetic, severe combined immunodeficiency, interleukin-2 receptor gamma chain-null) mice.

In all tumor models, tumor development was documented twice weekly by caliper measurements and volumes were calculated using the formula of (length ‘width’ width)/2. A week after tumor injection, or three days after receiving the TNBC PDX mice from Jackson Laboratories, mice were treated with vehicle (0.5% Avicel®), Gleevec (imatinib mesylate, 40 μg/g), or Pomiferin (5 μg/g) once daily by oral gavage administration. For HCC or PDAC xenograft models, mice were treated with vehicle (0.5% Avicel), Pomiferin (5 ug/g), and Compounds I, II, or III (5 ug/g). For the orthotopic syngeneic breast tumor model, inoculated mice were treated with vehicle (0.5% Avicel), Pomiferin (5 ug/g), or Compound III (5 ug/g). Mice were sacrificed according to the IACUC guidelines when tumor size reached two centimeters in diameter across a single dimension. Tumor material was harvested for both protein analyses by snap freezing in liquid nitrogen and histological studies by formalin fixation.

Mouse toxicity assays—Blood was collected from the submandibular facial vein following IACUC guidelines (CMP, Houston Methodist Research Institute and Rutgers University) before and 20-day after the drug treatment. The Department of Veterinary Medicine & Surgery, MD Anderson Cancer Center, performed the clinical biochemistry and pharmacokinetics tests. Blood serum chemistry analysis included alanine transaminase (ALT), aspartate transaminase (AST), blood urea nitrogen (BUN), and creatinine level measurements.

Statistical analysis—Each experiment was performed at least three times. The results are presented as the mean±standard error (SE). The significance of the differences between the mean values was assessed by a two-tailed Student's t-test using or ANOVA using GraphPad Prism 8.0 software (Graph-Pad Software; San Diego, CA). P<0.05 was considered statistically significant.

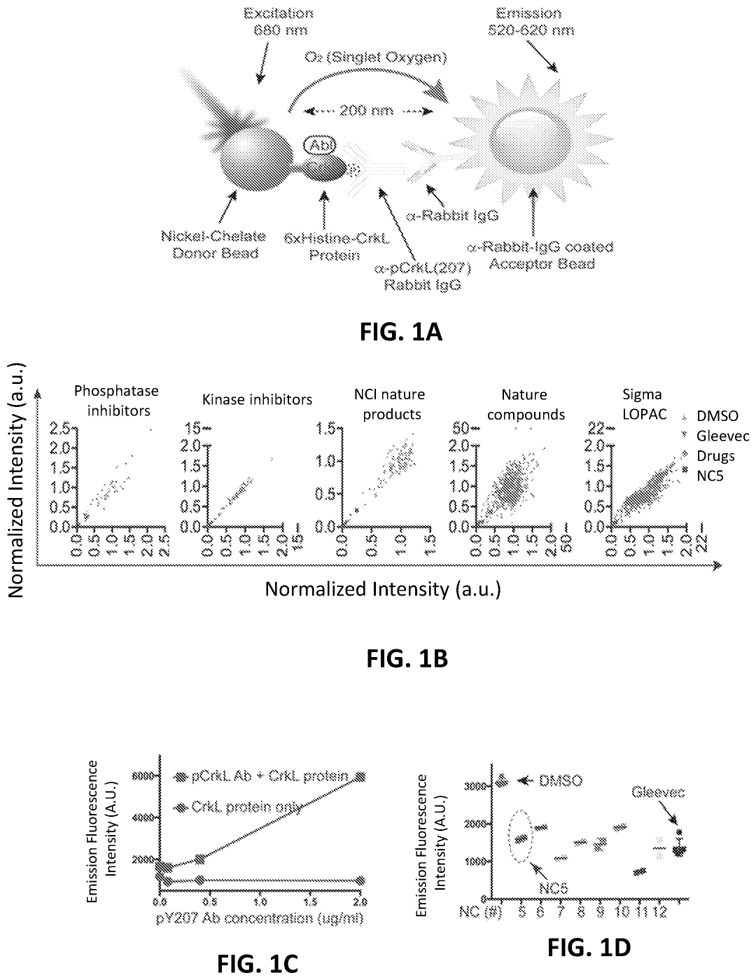

To identify specific chemical compounds that would inhibit phosphorylation of Y207 of CrkL, an AlphaScreen assay to screen five chemical compound libraries of potential inhibitors from The Center for Drug Discovery in Baylor College of Medicine (BCM) was designed. Nickel chelate donor beads were ligated to the six-histidine-tagged CrkL (6×His-CrkL). When 6×His-CrkL is phosphorylated by Ab1 kinase at Y207, 6×His-pCrkL can be detected by the specific anti-pCrkL antibody, Tyr 207 (rabbit IgG), which have been ligated to acceptor beads (FIG. 1A). When 6×His-pCrkL binds with the donor bead labeled with Tyr207 antibody, the distance between acceptor bead and donor bead becomes close enough to generate signals detected by a plate reader (FIG. 1A).

Specifically, when excited at 680 nm, the donor bead (nickel-chelated donor beads that can be conjugated with 6×His-CrkL fusion protein) produces singlet oxygen molecules. If the acceptor bead (anti-Rabbit-IgG coated acceptor bead) is within a distance of 200 nm from a donor bead, the singlet oxygen transfers energy to the donor bead resulting in the production of light between 520 and 620 nm. The amount of light produced is directly proportional to the amount of bound donor-acceptor beads. When a potential pCrkL inhibitor candidate was added into the reaction, this specific pCrkL inhibitor could bind to the CrkL phosphorylation site (Tyr207) directly, inhibiting the interaction between CrkL and Ab1, thus resulting in a reduced or absent CrkL phosphorylation signal. Using the AlphaScreen technique, 2312 drugs were screened and were categorized into five libraries (FIG. 1B): phosphatase inhibitors (# of compounds=33), kinase inhibitors (# of compounds=80), NCI natural product (# of compounds=119), nature compound (# of compounds=800), and Sigma LOPAC (# of compounds=1280).

To quantify the AlphaScreen data, the detected signal (production of light between 520 and 620 nm) from all screened compounds was normalized onto dot plots. Each compound was duplicated and plotted on both the x-axis and y-axis (FIG. 1B).

Compared to negative controls (DMSO treatment), positive hits were defined as a 50% decrease in Alpha signal. Gleevec (STI-571), a highly successful anti-cancer drug, inhibits Ab1, the upstream kinase of Crk. Gleevec was used as a positive control for this AlphaScreen assay. In the Gleevec treatment group (positive control), Alpha signal decreases of more than 80-90% were observed in the five libraries (FIG. 1B).

To further verify the AlphaScreen assay, different pY207 antibodies were used to detect the Alpha signal. A dose-dependent increase in Alpha signal was observed in this AlphaScreen assay (FIG. 1C). Thus, the data confirm that the anti-pCrkL antibody (shown with the symbol ▪) increases bead interaction in a concentration-dependent manner.

Using this technique, eight compounds (from the natural compound [NC] library) capable of inhibiting Crk phosphorylation (FIG. 1D) were identified; thus, it was concluded that the AlphaScreen design is valid and successful in the assay.

In the initial AlphaScreen assay, Gleevec served as a positive control. Inhibitors identified via the AlphaScreen assay will either: 1) inhibit Ab1 function (like Gleevec) or 2) block access to Tyr207 by precluding phosphorylation, which reduces CrkL phosphorylation signal. The latter could theoretically occur by a small molecule inhibitor binding to Ab1 or Crk; thus, a different assay was designed to distinguish between these two possibilities.

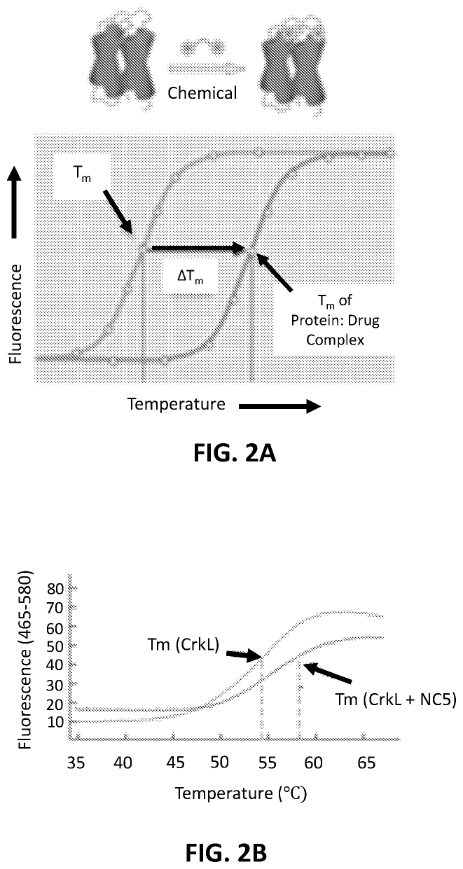

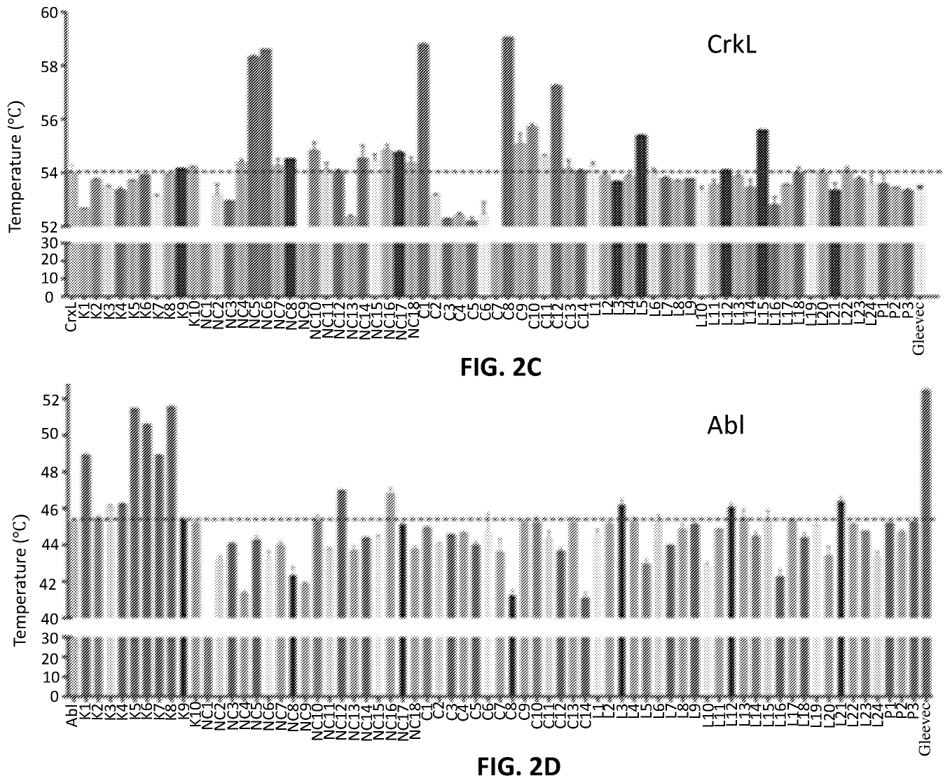

To identify specific pCrk inhibitors, thermal shift and in vitro kinase assays were used to distinguish between these possibilities. In a thermal shift assay, each tested protein (purified Ab1 or CrkL) has a melting temperature (Tₘ) detected by a protein stain dye such as Sypro Orange. After binding to a drug, the Tₘ of the tested protein will increase as a higher temperature is required to unfold the protein (FIG. 2A). A total of 71 candidate compounds from different libraries were evaluated, which were previously identified by the initial AlphaScreen assay, on both CrkL and Ab1. An increase of 2° C. or greater in Tₘ (stable increase in three independent experiments) was defined as a positive result in these thermal shift assays.

As illustrated in FIG. 2B, the Tₘ of CrkL increased, and the peak shifted to a high temperature. When NC5 was added to the reaction, regarding CrkL binding, five drugs (two from the natural compound library and three from the NCI natural product library) were also found to enhance protein Tₘ (FIG. 2C). Regarding Ab1 kinase binding (as a control), five drugs (all from the kinase inhibitor library) were found to significantly increase Tₘ in the thermal shift assay (FIG. 2D). Two compounds, K5 and K8, showed similar increases in Tₘ compared to the positive control Gleevec (Tₘ from 45° C. to 52° C., seven ° C. increase). NC5 from the natural compound library and C1 from the NCI natural product library share the same chemical name; NC6 (nature compound) is an isoform of C8 (NCI nature product). No single compound could bind to both Ab1 and CrkL proteins simultaneously based on the selection platforms; thus, NC5 is a promising candidate that specifically binds to Crk, but not Ab1 proteins.

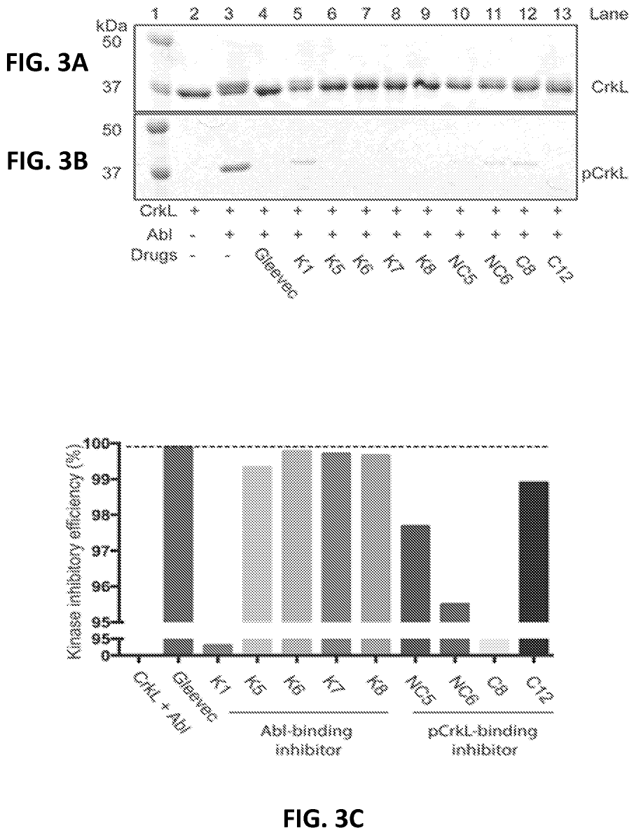

To further verify the pCrkL inhibitor, a kinase assay was designed to evaluate the specificity of positive hits. The in vitro kinase assay provides a practical way to verify the role of candidate compounds on CrkL phosphorylation. In this assay, CrkL proteins can be phosphorylated by Ab1 kinase in the presence of ATP at 30° C. When adding drugs (such as Gleevec) to the reaction, the interaction between Ab1 and CrkL is blocked, thus decreasing the amount of phosphorylated CrkL. The decreased amount of pCrkL can be detected and quantified by this kinase assay. Here, the modified kinase assays were used to confirm that the candidate molecules do indeed inhibit CrkL phosphorylation. FIG. 3A shows a representative western blot. Note that NC5 inhibits Ab1 Phosphorylation at the 37kD CrkL band.

In total, ten candidate drugs (five that bind to Ab1 and five that bind to CrkL) were verified by the in vitro kinase inhibition assay followed by thermal shift analysis. As illustrated in FIGS. 3A and 3B, both CrkL and phosphorylated CrkL was detected separately by western blot analysis after the in the vitro kinase assay. For total CrkL detection, the antibody, C20, was used in this experiment to detect the phosphorylated CrkL that is a band shift from the non-phosphorylated CrkL (FIG. 3A, shown at the higher molecular weight). For phosphorylated CrkL detection, only the phosphorylation of Tyr207 was specifically detected by the anti-CrkL phosphorylation antibodies (FIG. 3B). In FIG. 3B, different band signals of the pCrkL were observed after drugs were bound to CrkL proteins, indicating that these candidate drugs have different abilities to bind and inhibit CrkL phosphorylation. Compared to Gleevec (a positive control), drugs binding to Ab1 (e.g., K5, K6, K7, and K8) and drugs binding to CrkL (NC5 and C12) showed comparable results similar to the kinase inhibition effect seen with Gleevec. Based on the results of the AlphaScreen assay, thermal shift assay, and in vitro kinase assay (results shown in FIG. 3C), NC5 was selected for further studies.

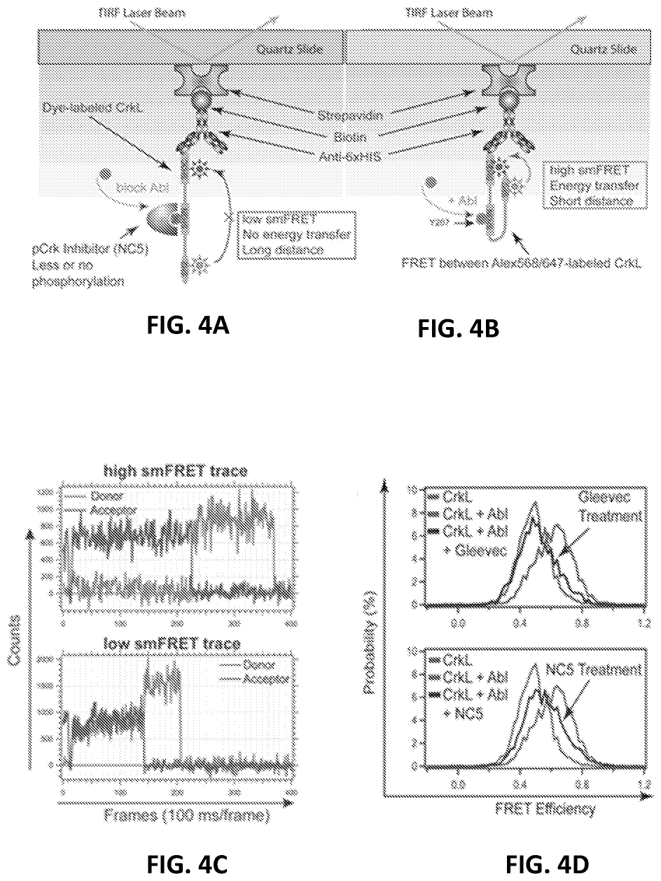

In intramolecular smFRET, non-radiative energy transfer occurs when two fluorophores attached to different parts of one molecule move close together. Previous studies show that pCrk exists in a closed molecular conformation, whereas non-phosphorylated Crk exists in an open conformation, which allows smFRET changes to report the phosphorylation state (see schematic summary in FIGS. 4A and 4B). A cysteine-maleimide-based chemistry was used to label both CrkL and mutant (non-phosphorylatable) CrkL-Y207F. CrkL has a cysteine (a relatively rare amino acid) in its SH2 (labeled with AlexaFluor555) and SH3C (labeled with AlexaFluor647). When CrkL is phosphorylated, FRET is high (FIG. 4D, line labeled “CrkL+Ab1”); when de-phosphorylated, FRET decreases (FIG. 4, line labeled “CrkL+Ab1+Gleevec”); (and these are consistent with the “open” and “closed” states mentioned above). Mutation to a non-phosphorylatable form of CrkL (Y207F) prevents a shift to the open molecular conformation (data not shown). Similarly, a specific inhibitor of Crk phosphorylation would also be expected to result in decreased FRET ratio (FIG. 4C). As shown in smFRET histograms in FIG. 4C, the addition of Ab1 in the presence of Mg²⁺ and ATP to increase pCrkL led to higher FRET, inhibited by either Gleevec or NC5 (55%). The CrkL-Y207F mutant did not show a shift in smFRET (data not shown). Western Blot (WB) and immunoprecipitation (IP) was used to verify that labeled proteins retain the same gross functional properties as wild-type CrkL (e.g., are phosphorylatable and capable of binding SH2 and SH3 ligands). Briefly, site-specific labeled CrkL was mixed with Ab1 in the presence of Mg²⁺ and ATP. A single AlphaScreen-identified candidate inhibitor was then added per assay. Inhibition of Crk phosphorylation resulted in decreased FRET in a concentration-dependent manner.

To confirm that NC5 can inhibit CrkL phosphorylation, the smFRET assay was applied to detect conformational changes due to protein phosphorylation after binding by the drug. As shown in FIG. 4D, the FRET signal of CrkL shifted from a non-phosphorylated status (red line labeled “CrkL”, about 0.4) to a phosphorylated status (line labeled “CrkL+Ab1”, about 0.6) when adding Ab1 protein. The reaction with Gleevec added inhibits CrkL phosphorylation and shifts back the FRET signal to 0.4 (FIG. 4D, line labeled “CrkL+Ab1+Gleevec”). Therefore, Gleevec was used as a positive control. The effect of NC5 on phosphorylated CrkL conformational changes was detected. That data show that NC5 can also shift the FRET signal back to the lower state (from 0.6 to 0.5) of CrkL by blocking the phosphorylation (FIG. 4D, black line). Altogether, NC5 was found to directly bind to the CrkL protein and inhibit CrkL phosphorylation in vitro. As such, Compounds I-V also are believed to exhibit similar binding behavior.

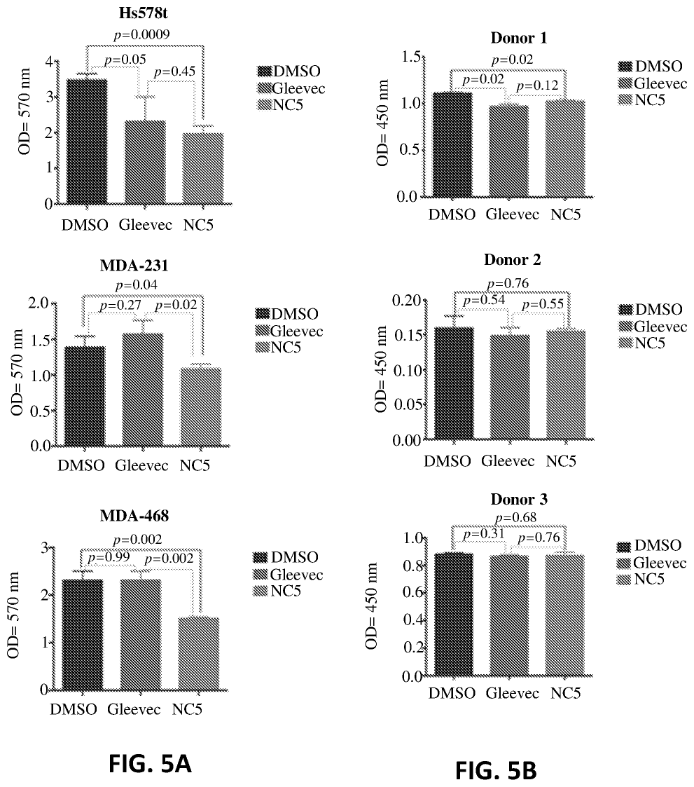

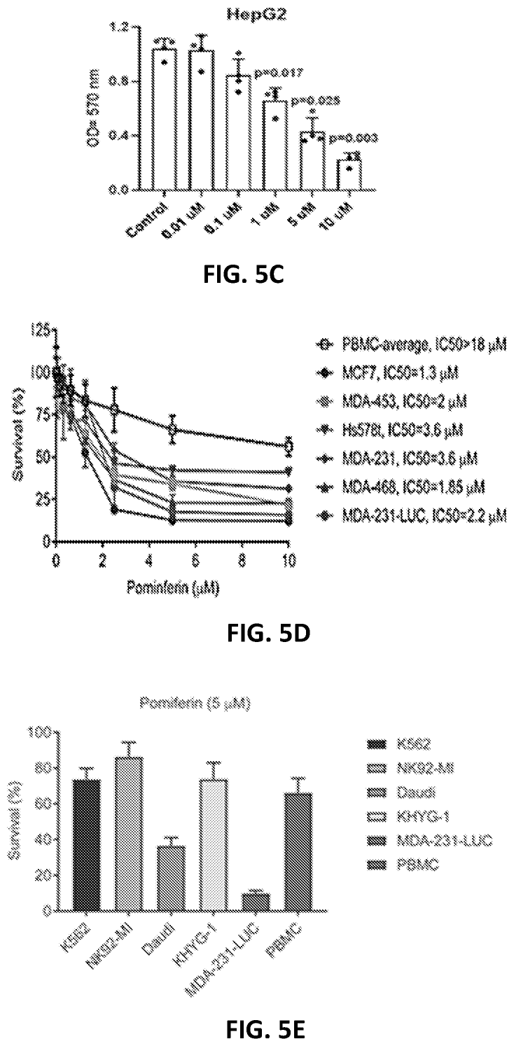

Next, the in vivo function of Pomiferin was further evaluated by using a cell-based system. Given that CrkL is over-expressed in breast cancers, the effects of Pomiferin on three breast cancer cell lines was evaluated: MDA-MB-231, MDA-MB-468, and Hs578T. These three breast cancer cell lines were treated with 2.5 μM Gleevec (control group), 2.5 μM Pomiferin, and 1:4000 dilution of DMSO (vehicle group). In the Hs578T cell group, both Gleevec (p<0.05) and Pomiferin (p<0.01) had an inhibitory effect on proliferation, but no significant difference between Gleevec and Pomiferin was observed (FIG. 5A, upper left panel). In the MDA-MB-231 cell group, only Pomiferin treatment blocked cell proliferation (p<0.05), while Gleevec did not affect MDA-MB-231 cell proliferation (FIG. 5A, middle left panel). In the MDA-MB-468 cell group, a similar result was obtained in both Pomiferin-treated (p<0.02) and Gleevec-treated cells (FIG. 5A, lower left panel). Therefore, at a concentration of 2.5 M, Pomiferin significantly inhibited breast cancer cell line proliferation. As such, Compounds I-V also are believed to exhibit similar inhibitory behavior.

Previous studies have shown that phosphorylated CrkL inhibits immune cell functions. It was then evaluated whether a pCrkL inhibitor could restore immune cell functions. To test this, the production of IL-2 from human PBMCs treated with different concentrations of Pomiferin was quantitated. Phytohemagglutinin (PHA) was used to stimulate the secretion of IL-2 as a positive control group. Compared to DMSO (vehicle group) from the first donor, decreased IL-2 production from PBMCs treated with Pomiferin and Gleevec was observed. However, no differences between Pomiferin and Gleevec treatment were observed (FIG. 5B, upper right panel). No difference was observed from the second donor between DMSO and Gleevec, DMSO and Pomiferin, and between Gleevec and Pomiferin (FIG. 5B, middle). Similar results were obtained from the third donor (FIG. 5B, lower panel).

In conclusion, although Pomiferin does not enhance normal immune cell functions, it does not inhibit immune cell function either; thus, the data show that Pomiferin alone inhibits breast cancer cell line proliferation but may not have any detectable effects on immune cell function using IL-2 as a readout.

To further elucidate the specificity of killing effects of Pomiferin on breast cancer cells rather than immune cells, the survival percentage was calculated at different concentrations of Pomiferin treatment between breast cancer cells, PBMCs from healthy donors, HCC-HepG3 cells, and leukemia cells using MTT proliferation assays.

Pomiferin not only could inhibit cell proliferation in breast cancer cells but also had a dramatic growth inhibition effect on the other solid tumor HCC HepG2 cells, HepG2 cells treated with various concentrations of Pomiferin for 48 hours, it started to significantly inhibit HepG2 cell proliferation at one M (FIG. 5C). These results suggest the broad potential use of Pomiferin for various tumor cell lines besides breast cancer cells.

Different subtypes of breast cancer, including non-triple negative breast cancer (non-TNBC) cell lines (MCF7 and MDA-MB-453) and TNBC (MDA-MB-231, MDA-MB-231-GFP-LUC, Hs578t, and MDA-MB-468) cell lines, were included in the assay (FIG. 5D). The average percentage survival of PBMCs from four different healthy donors was significantly higher than that of the breast cancer cell lines treated with different concentrations of Pomiferin. Meanwhile, The IC50 of the breast cancer cells was range from 1.3 to 3.6 μM and was >18 μM in PBMCs, which indicates the specificity of Pomiferin to inhibit breast cancer cell survival and further indicates that Compounds I-V would exhibit similar behavior.

To further test the inhibitory specificity of Pomiferin for specific breast cancer cell lines, The survival percentage was compared between different hematopoietic cancer cell lines and healthy PBMCs. K562, NK92-MI, Daudi, KHYG-1, PBMCs, and MDA-231-GFP-LUC cells were treated with five M Pomiferin for three days. The hematopoietic cancer cell lines (K562, NK92-MI, Daudi, and KHYG-1) and the normal PBMCs were more resistant to Pomiferin inhibition than the MDA-MD-231-LUC breast cancer cell line, which was the most sensitive cell to Pomiferin treatment (FIG. 5E). This data shows that Pomiferin has better killing efficacy on the breast cancer cells than the hematopoietic cancer cell (FIG. 5E). Overall, the natural compound Pomiferin can specifically inhibit breast cancer cell proliferation in vitro.

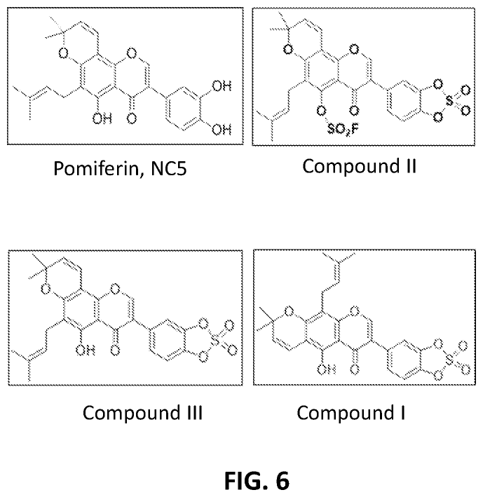

In view of the data obtained for Pomiferin, Compound II, Compound III, and Compound I (FIG. 6) were synthesized to measure the biological activities towards various tumor cells.

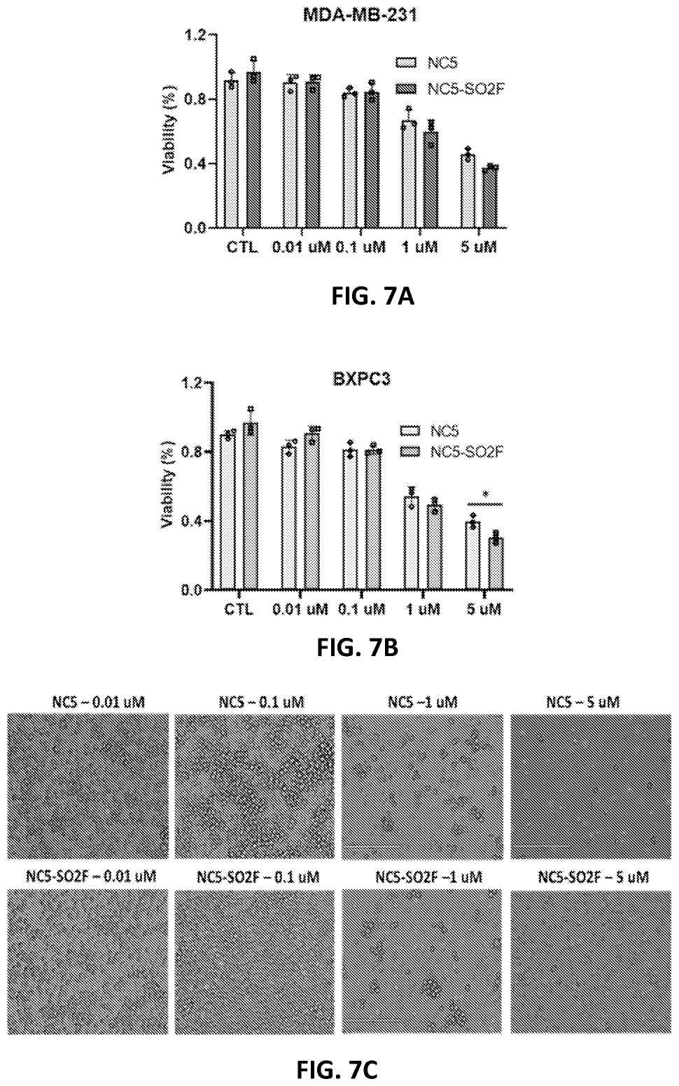

To compare the killing effects of compound II with NC5, survival percentage was compared of different concentrations of compound II treatment between breast cancer cells MDA-MB-231 and PDAC cells BXPC3 using MTT proliferation assays. In some examples, Compound II showed a comparable killing effect as NC5 in MDA-MB-231 cells (FIG. 7A). While in BXPC3 cells, Compound II had a stronger killing effect than NC5 at 5 uM (FIG. 7B). This killing effect was further confirmed by propidium iodide staining of BXPC3 cells treated with various concentrations of NC5 or Compound II (FIG. 7C). Compound II is referred to as “NC5-SO2F” in FIGS. 7A-7C.

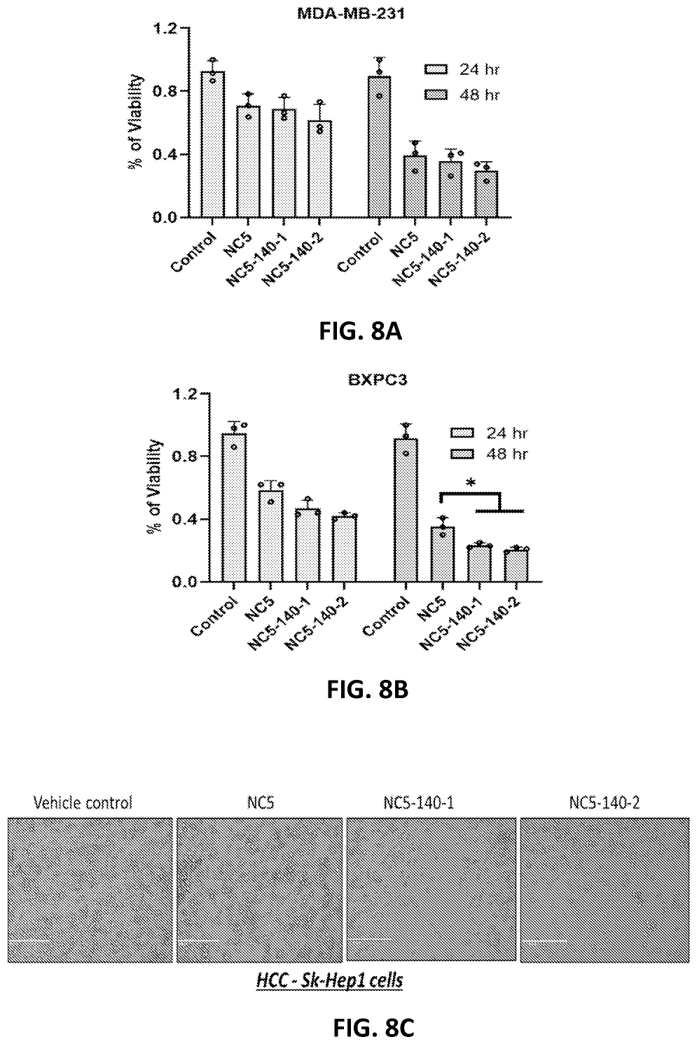

Compound embodiments, compound III and compound I, were also made and evaluated. TNBC cells and PDAC cells were treated with a serial dose of original NC5 and modified NC5 derivatives for 24 and 48 hours. The cell viability will be measured by XTT assay. Both Compound III and Compound I had a comparable toxic effect as NC5 in MDA-MB-231 cells (FIG. 8A). They also showed a better toxic effect than NC5 when used in BXPC3 (FIG. 8B) and Sk-Hep1 cells (FIG. 8C). Compound III is referred to as “NC5-140-1” and Compound I is referred to as “NC5-140-2” in FIGS. 8A-8C.

To further test the specificity of NC5 in vivo, a breast cancer cell line xenograft model was used. Several groups have used an orthotopic xenograft TNBC animal model using MDA-MB-231-GFP-LUC cells to demonstrate the efficacy of in vivo tumor inhibition. Specifically, luciferase- and GFP-expressing MDA-231-GFP-LUC cells were implanted into the mammary fat pads of NSG™ mice. Six days after tumor cell implantation, the initial tumor establishment was confirmed by bioluminescent imaging using In Vivo Imaging Systems (IVIS). After confirmation, then the mice were randomly divided into three treatment groups: (1) vehicle control group (0.5% Avicel®), (2) Gleevec treatment group, (3) Pomiferin treatment group. The treatment was administered through oral gavage administration at a dose of 0.5% Avicel® for the vehicle control group, 40 μg/g body weight for the Gleevec-treatment group, and 5.0 μg/g body weight for Pomiferin, respectively. The Pomiferin treatment dose for the Pomiferin-treatment group was based on previous literature showing that a dose of 5 mg/kg/day in 0.5% Avicel® achieved cardio-protection and improved ventricular function. This example also illustrates the in vivo titration analysis, indicating that the dose range from 3.125-6.25 mg/kg/day significantly inhibited breast cancer growth in mice (data not shown).

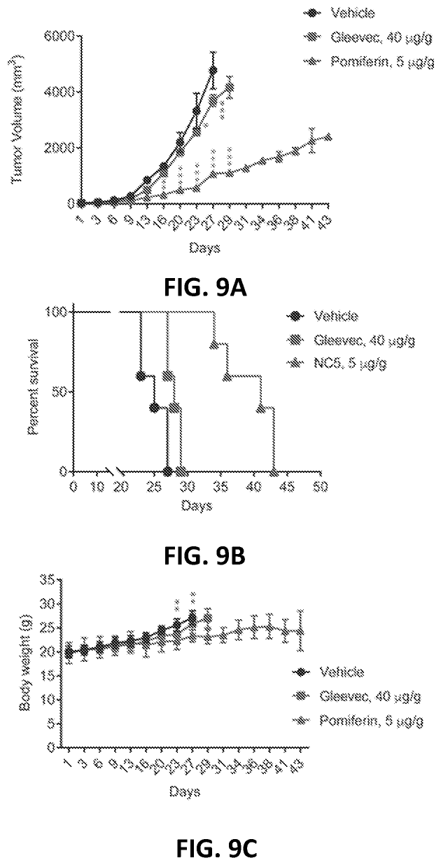

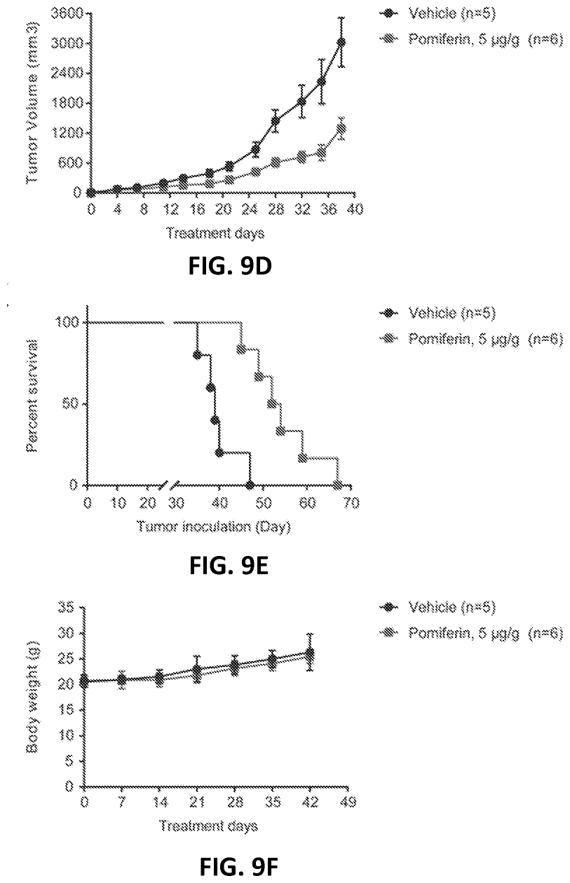

In the MDA-MB-231-GFP-LUC orthotopic xenograft model, a 5.0 mg/kg/day Pomiferin treatment dose was used, compared with the eight-fold higher dose of the currently FDA-approved drug Gleevec (also known as imatinib mesylate) (40 mg/kg/day). It was observed that 5.0 mg/kg/day of Pomiferin in an orally once daily delivered regimen significantly suppressed MDA-MB-231 tumor growth in mice, which represents the most aggressive TNBC subtype (FIG. 9A). Low dose Pomiferin treatment also prolonged mouse survival compared to vehicle control and the 8-fold higher dose of the Gleevec treatment group. The median survival in days of the Pomiferin-treated group (41 days) was 1.6-fold longer than the vehicle control (25 days) and 1.4-fold longer than the Gleevec treatment group (28 days), as shown in FIG. 9B.

To quantify the toxicity of Pomiferin, mouse body weight was monitored throughout drug treatments. No significant difference was observed among the three treatment groups until the end-stage (in 23 to 27 days, FIG. 9C). The increased body weight difference between the groups after day 23 was caused by the large tumor growth in the vehicle and Gleevec treatment mice (each tumor weight was 1.8-2 grams, data not shown).

Together, the low dose of Pomiferin therapy significantly inhibits MDA-MB-231 tumor growth in vivo without noticeable adverse effects.

To further confirm the breast cancer cell line model can recapitulate the human breast cancer model, a patient-derived xenograft (PDX) model was used, since Pomiferin treatment effectively suppressed MDA-MB-231 tumor growth in mice. To determine inhibition efficacy in vivo of Pomiferin in the TNBC clinical patient tumor, the therapeutic efficacy of Pomiferin in a patient-derived tumor xenograft mouse from the Jackson Laboratory PDX Live™ library was evaluated. The TM00089 breast invasive ductal carcinoma was selected, characterized as TNBC ER⁻/PR⁻/HER2⁻, BRCA1^{V757fs} since TNBC tumors with BRCA1 mutations are generally the most aggressive and have the worst prognosis within the first five years after diagnosis compared to other types of breast cancers. Consistent with the previous result in the TNBC cell-tumor-bearing mice, TNBC-patient-derived tumor growth was significantly more inhibited in 5.0 μg/g Pomiferin-treated mice than vehicle control-treated mice (FIG. 9D).

The median survival of the Pomiferin-treated mice (53 days) was 1.4-fold longer than the vehicle control (39 days, p=0.0025) (FIG. 9E). The body weights of the vehicle- and Pomiferin-treated mice were not significantly different during the drug treatment period (FIG. 9F). These results demonstrated that Pomiferin therapy could efficiently repress TNBC growth in the two different xenograft breast cancer models (MDA-MB-231 cells and TNBC patient tumor segments).

To further test whether the tumor growth inhibition effect of NC5 applied to other solid tumors, the HCC-Sk-Hep1cell line and PDAC-BXPC3 cell line xenograft models were used. Subcutaneous injection of 1.5×10⁶ SK-Hep1 or BXPC3 cells in NSG mice, Six-ten days after tumor cell injection, the palpable tumor in NSG mice was confirmed. After confirmation, the mice were randomly divided into two treatment groups: (1) vehicle control group (0.5% Avicel), (2) Pomiferin treatment group. The treatment was administered through oral gavage administration at a dose of 0.5% Avicel for the vehicle control group, 5.0 μg/g body weight for the Pomiferin group.

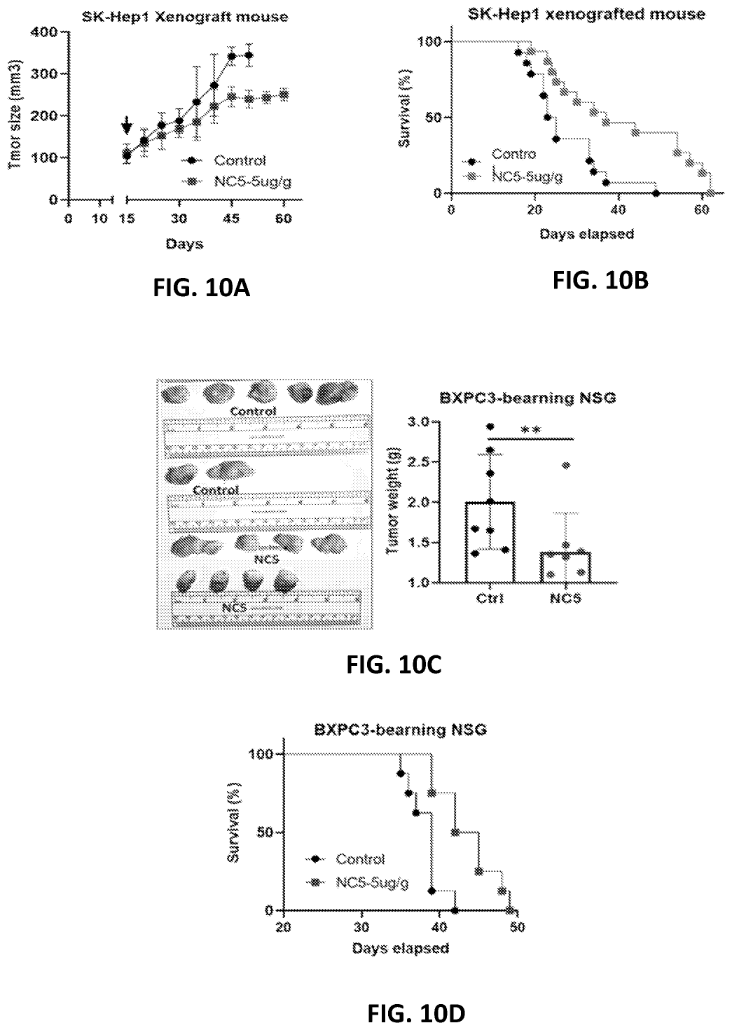

Tumor sizes and body weight were measured every 3-4 days. It was observed that 5.0 g/g/day of Pomiferin administered orally suppressed Sk-Hep1 and BXPC3 tumor growth in mice, similar to that of the TNBC tumor model (FIGS. 10A and 10C). Pomiferin treatment also prolonged mouse survival compared to those treated with vehicle control in both xenograft models (FIGS. 10B and 10D). The median survival of the Pomiferin-treated mice (37 days) was 1.54-fold longer than the vehicle control in the Sk-Hep1 tumor model (24 days, p=0.0045). The median survival of the Pomiferin-treated mice (43.5 days) was 1.2-fold longer than the vehicle control in the BXPC3 tumor model (37 days, p=0.0025).

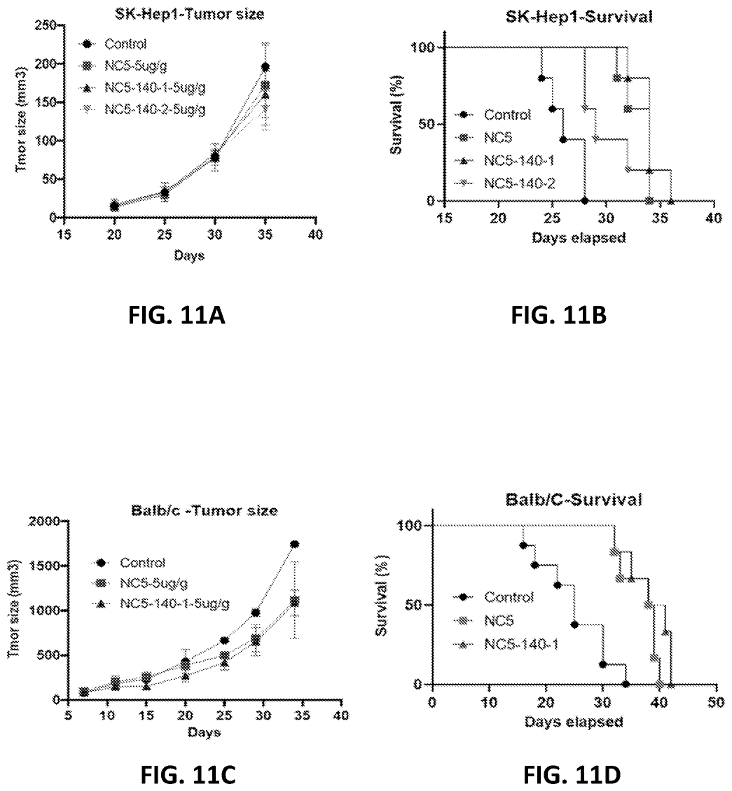

To test the effects of the disclosed compounds on the xenograft mouse models, the HCC-Sk-Hep1 cell line xenograft model in NSG mice and 4T1-Luc orthotopic syngeneic breast tumor model in Balb/c mice was used. After confirming the palpable tumor in NSG or Balb/c mice. The mice were then randomly divided into four treatment groups: (1) vehicle control group (0.5% Avicel), (2) NC5, 5.0 μg/g, (3) Compound III, 5.0 μg/g, (4) Compound I, 5.0 μg/g; Balb/c mice into three groups: (1) vehicle control group (0.5% Avicel), (2) NC5, 5.0 μg/g, (3) Compound III, 5.0 μg/g, the treatment was administered through oral gavage administration at a dose of 0.5% Avicel for the vehicle control group, 5.0 μg/g body weight for treatment groups. Compound III is referred to as “NC5-140-1” and Compound I is referred to as “NC5-140-2” in FIGS. 11A-11C.

Tumor sizes and body weight were measured every 3-4 days. There was no significant difference in mice body weight among the treatment and control groups (data not shown). NC5 and compound embodiments of the present disclosure administered orally showed no significant difference in tumor sizes before day 25 of inoculation of Sk-Hep1 tumor cells. An inhibition of tumor growth was observed in all treatment groups compared to control groups (FIG. 11A). The median survival of the NC5-treated mice (34 days), Compound III (34.5 days), and Compound I (28.5 days) were all longer than the vehicle control in the Sk-Hep1 tumor model (26 days).

In the orthotopic syngeneic 4T1 mammary fat pad Balb/c model, steady growth inhibition of both NC5 and Compound III (FIG. 11C) was observed. The median survival of the NC5-treated mice (38.5 days) and Compound III (39.5 days) was significantly longer than the vehicle control in the 4T1/Balb/c tumor model (25 days, p=0.002) (FIG. 11D). In conclusion, Compound III is a promising anti-cancer compound against solid tumors.

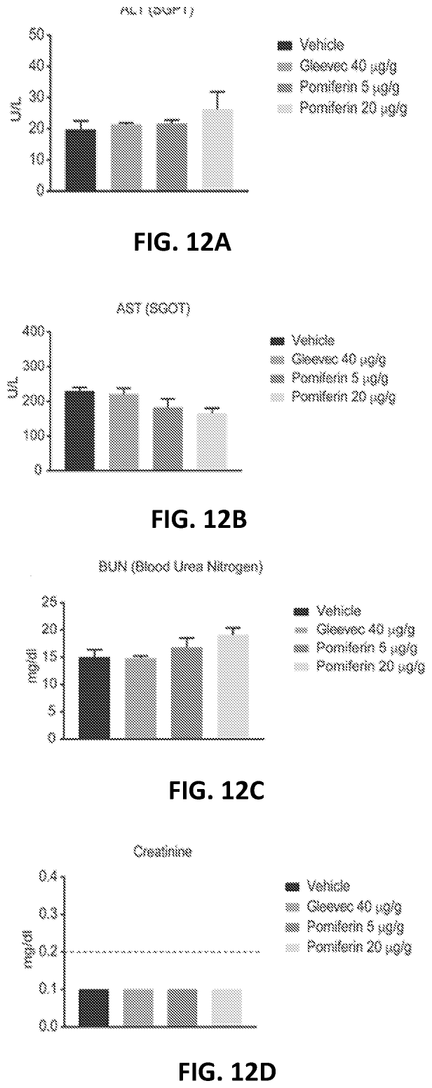

Alanine aminotransferase (ALT, SGPT), aspartate aminotransferase (AST, SGOT), blood urea nitrogen (BUN), and creatinine levels were examined to determine liver and kidney toxicity of Pomiferin treatments in vivo. The mice were treated with low dose Pomiferin (5 μg/g) or high dose Pomiferin (20 μg/g), side-by-side with Gleevec (imatinib mesylate, 40 μg/g) and vehicle (0.5% Avicel). The high dose (20 μg/g) Pomiferin-treated group showed slightly elevated ALT (FIG. 12A), decreased AST (FIG. 12B), and slightly increased blood urea nitrogen (BUN) levels (FIG. 12C). Nevertheless, these changes have no physiological significance because ALT, AST and BUN levels in all mice were within normal mouse reference ranges. The blood level of creatinine in the four treated groups was all below 0.2 mg/dl (FIG. 12D).

Overall, these results indicate that Pomiferin therapy and Gleevec, at the indicated dosages, produce little to no damage to the liver and kidneys during treatment in mice.

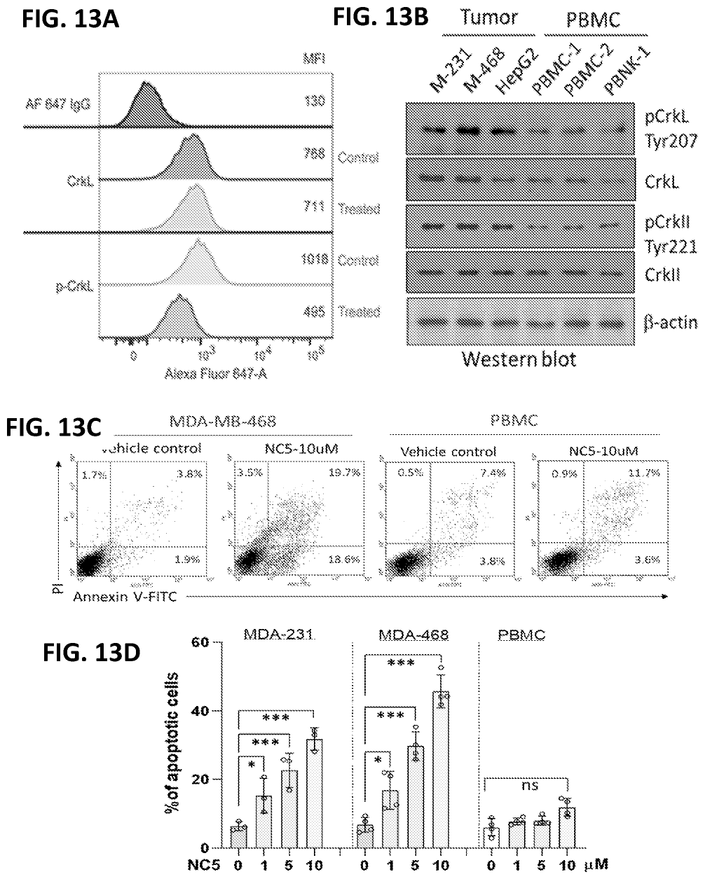

To demonstrate the specificity of Pomiferin on CrkL. The intracellular pCrkL level from breast cancer cell lines between Pomiferin-treated and vehicle control groups was examined. As expected, Pomiferin-treated MDA-MB-231-LUC cells reduced the intracellular pCrkL level (FIG. 13A) compared to vehicle-treated cells by flow cytometry analysis. There are not many differences in total CrkL expression following Pomiferin treatment (FIG. 13A, middle panel).

To investigate the different responses of tumor or normal cells to NC5, the basal protein levels of pCrkL and CrkL were measured by Western blots with MDA-231, MDA-468 and HepG2 cells. PBMC cells from normal donors were used along as a comparison. The tumor cells have much higher basal levels of pCrkL than normal PBMC, which could be the base for the specificity of NC5 on inhibition of pCrkL (FIG. 13B).

To figure out the features of cytotoxic effects of Pomiferin on breast cancer cells. Pomiferin-treated breast cancer cells were stained with an Annexin V/PI kit and measured them with Flow Cytometry. The experiments were repeated at least three times in MDA-MB-231, MDA-MB-468 and PBMC cells. The percentage of apoptotic cells was significantly increased by treating 10 uM of NC5 for 24 hours in MDA-MB-468 cells. PBMC cells showed limited responses to NC5 treatment (FIG. 13C). The apoptotic effects of NC5 in both MDA-231 and MDA-468 cells were summarized in the bar chart; even the low concentration of NC5 (1 uM) significantly induced apoptosis in TNBC (FIG. 13D).

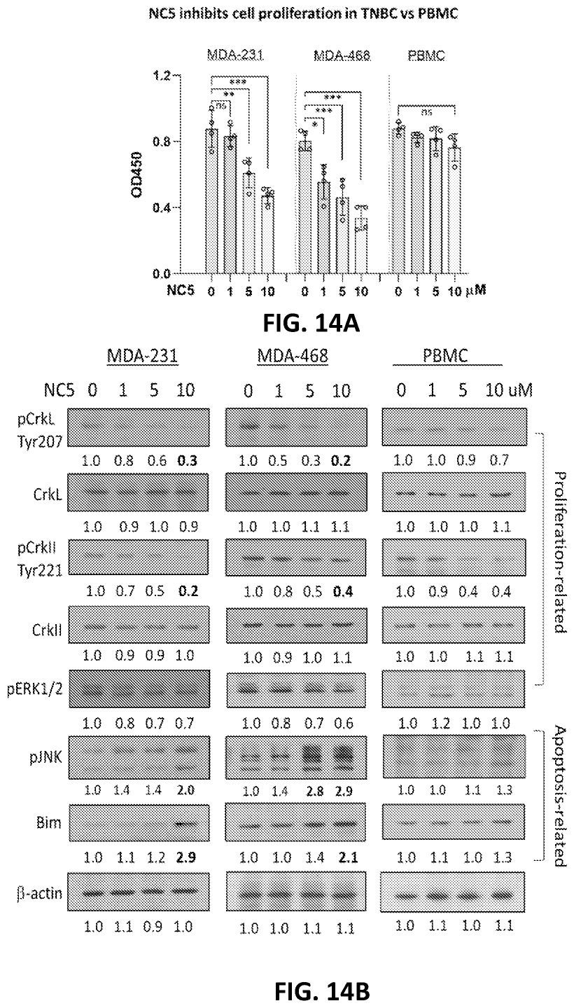

To investigate the molecular mechanisms of Pomiferin-induced apoptosis in breast cancer cells. MDA-231, MDA-468 and PBMC cells were treated with NC5 (1 uM, 5 uM and 10 uM) for 24 hours. Cell proliferation was first determined by MTT assay as confirmation. NC5 significantly inhibited cell proliferation in MDA-231 and MDA-468 cells, but no statistical difference in PBMC isolated from normal donors (FIG. 14A). Then the protein levels of pCrkL, CrkL, cell proliferation- and apoptosis-related targets were determined by Western blotting. pCrkL levels were dramatically inhibited by NC5 treatment. CrkL is known to mediate the activation of both Raf/ERK and PI3K pathways. So, proteins that relate to these two pathways were utilized. Inhibition of pCrkL reduces P-ERK1/2 levels in breast cancer cells compared to minimal changes in PBMC.

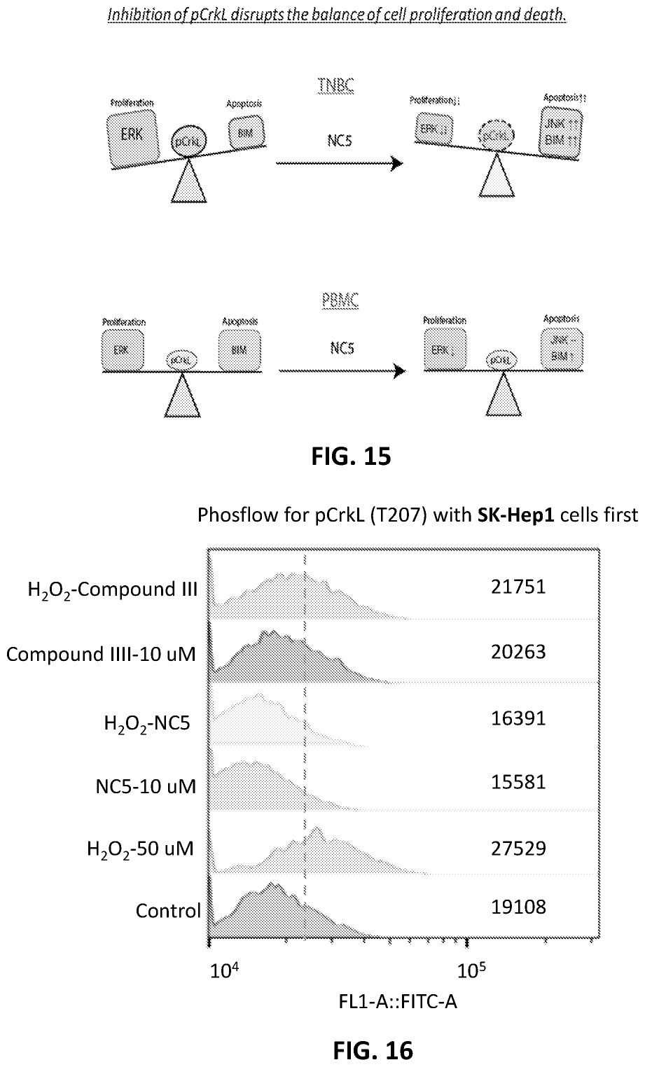

The apoptotic related proteins, pJNK and BIM, levels also were determined and were dramatically increased by NC5 and correlated to the enhancement of apoptosis in cancer cells (FIG. 14B). β-actin was used as a control to normalize loading. The Integrated Density Values (IDV) of each band were measured and shown under each blot. These data suggest that ERK1/2 and BIM are the key target proteins related to the inhibition of pCrkL by Pomiferin. Inhibition of pCrkL breaks the balance of cell proliferation and death in triple-negative breast cancer cells by regulating these signaling transduction pathways (FIG. 15). The different expression of these proteins in TNBC vs. normal immune cells makes Pomiferin and compounds disclosed herein a candidate for cancer therapy.

### Example 3

In this example, solubility measurement assays were performed using a phosphate buffer (also referred to as “PB”) system and a water system to evaluate thermodynamic solubility. The PB system was at a pH of 7.4 to mimic in vivo environments. For the PB system, the values were determined to be NC5: Thermodynamic Solubility pH=7.4<0.42 μg/mL; Compound III: Thermodynamic Solubility pH=7.4<0.48 μg/mL; and Compound I: Thermodynamic Solubility pH=7.4<0.48 μg/mL. Results for the water assay were as follows: NC5: Thermodynamic Solubility Water<0.42 μg/mL; Compound III: Thermodynamic Solubility Water<0.48 μg/mL; and Compound I: Thermodynamic Solubility Water<0.48 μg/mL.

### Example 4

In this example, Sk-Hep1 cells were pretreated with 50 uM H₂O₂ for 1 hour to induce activation of pCrkL and were then incubated with 10 uM NC5 or Compound III for 24 hours. pCrkL (T207) expression was determined by flow cytometry. Both NC5 and Compound III inhibited H₂O₂ induced pCrkL protein levels in Sk-Hep1 cells, as shown by FIG. 16.

### Example 5

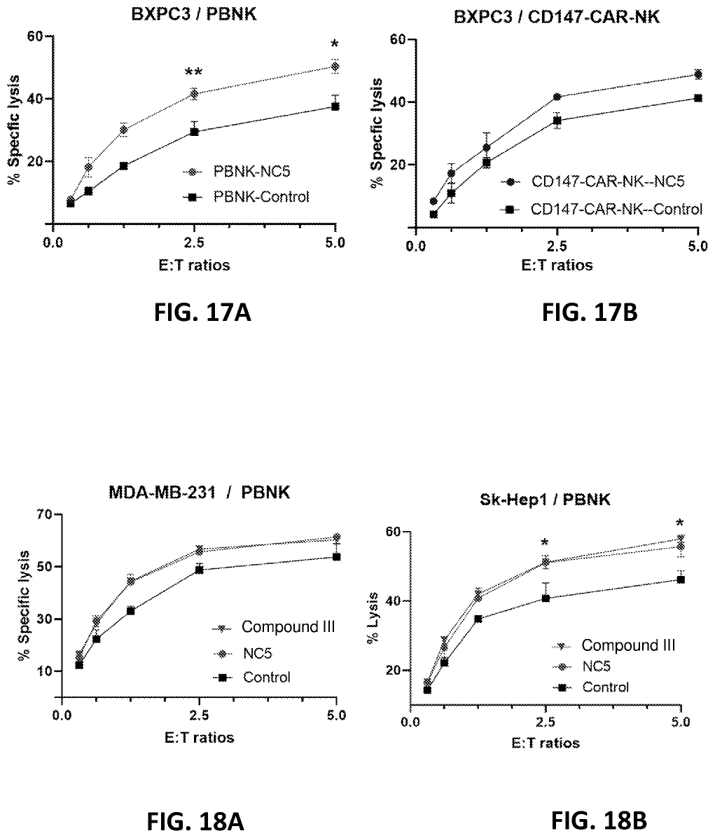

In this example, activity of NC5 was evaluated for PBNK and CD147-CAR-NK killing in BXPC3 cells. PBNK or CD147-CAR-NK cells were incubated with 1 uM NC5 overnight, then cells were harvested for Cr51 release assay to check cell cytotoxicity. As can be seen in FIGS. 17A and 17B, increased PBNK (FIG. 17A) and CD147-CAR-NK (FIG. 17B) killing in BXPC3 cells was observed for NC5. The star symbol indicates the significance of vehicle vs. NC5 of the indicated E:T ratios. *p<0.05, **p<0.01, n=3.

### Example 6

In this example, activity of NC5 and Compound III were evaluated for PBNK cells killing activity towards MDA-MB-231 and SK-Hep1. PBNK cells were incubated with 1 uM NC5 or Compound III overnight, then cells were harvested for Cr51 release assay to check cell cytotoxicity. As can be seen in FIGS. 18A and 18B, NC5 and Compound III increased PBNK cells killing activity towards MDA-MB-231 (FIG. 18A) and SK-Hep1 (FIG. 18B). The star symbol indicates the significance of vehicle control vs. NC5 of the indicated E:T ratios. *p<0.05, **p<0.01, n=3.

### Example 7

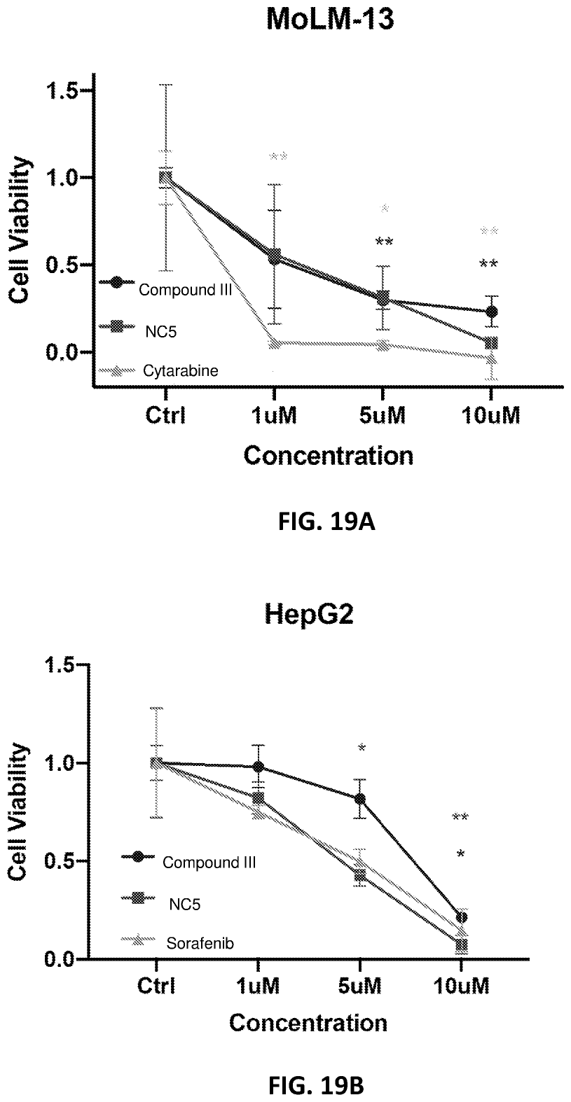

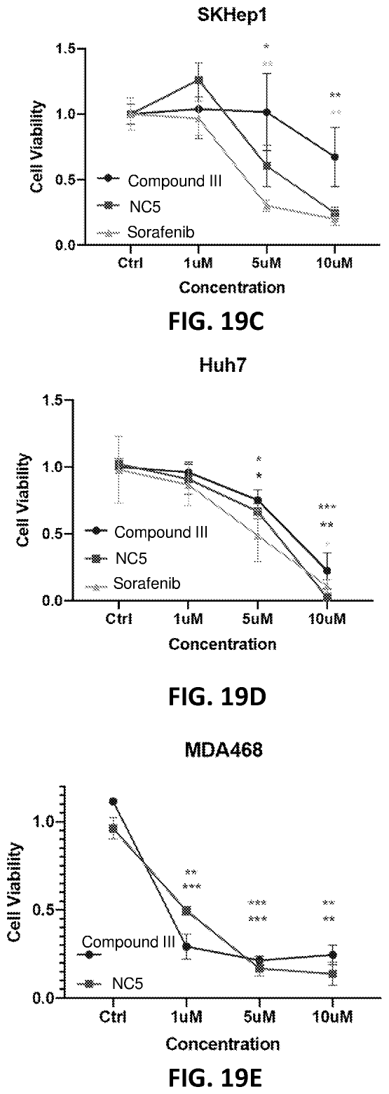

In this example, activities of NC5, Compound III, and the known chemotherapeutic, Cytarabine, were evaluated in the AML cell line (MoLM-13). MoLM-13 cells were collected and seeded at 5,000 cells/well in 96-well plates. The cells were incubated for 24 hours before the addition of either DMSO control or drug at various concentrations. After an additional 48 hours of incubation, 3 μL of CCK-8 was added to every well and incubated at 37° C. for 3 hours. Absorbance was measured at 460 nm in a plate reader. The AML cells treated with NC5 and Compound III at various concentrations (1 uM, 5 uM, 10 uM) show decreased cell viability via CCK-8 assay. The related absorbance was normalized and plotted as line graphs. p-values were determined through two tails student t-tests (*=p<0.5, **=p<0.01, ***=p<0.001). As shown in FIG. 19A, Compound III decrease cell viability significantly at different concentrations for AML tumor cell lines.

### Example 8

In this example, activities of NC5, Compound III, and the known chemotherapeutic, Sorafenib, were evaluated in HepG2, SKHep1, Huh7, and MDA468 cell lines. Cells from each cell line were collected and seeded at 5,000 cells/well in 96-well plates. The cells were incubated for 24 hours before the addition of either DMSO control or drug at various concentrations. After an additional 48 hours of incubation, 3 μL of CCK-8 was added to every well and incubated at 37° C. for 3 hours. Absorbance was measured at 460 nm in a plate reader. Cells from each cell line were treated with NC5 and Compound III at various concentrations (1 uM, 5 uM, 10 uM). The related absorbance was normalized and plotted as line graphs. p-values were determined through two tails student t-tests (*=p<0.5, **=p<0.01, ***=p<0.001). As shown in FIGS. 19B-19E, Compound III decreased cell viability significantly at different concentrations in each cell line.

In view of the many possible embodiments to which the principles of the present disclosure may be applied, it should be recognized that the illustrated embodiments are only preferred examples of the disclosure and should not be taken as limiting the scope of the disclosure. Rather, the scope is defined by the following claims. We therefore claim as our invention all that comes within the scope and spirit of these claims.

1. A compound selected from

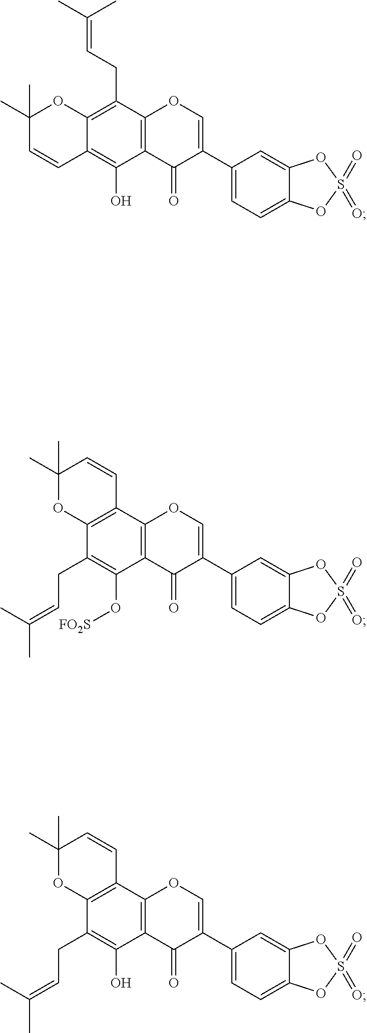

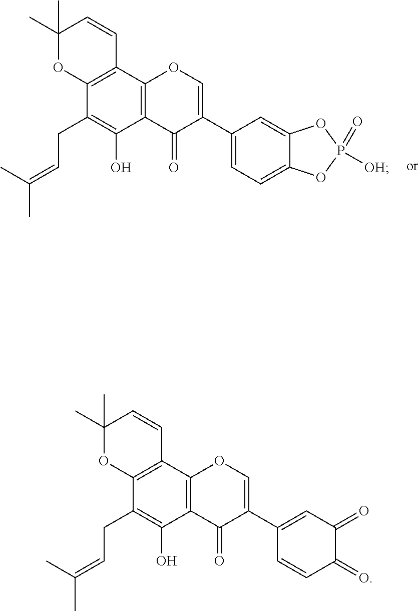 [SMILES: CC(C)=CCc1c2c(c3occ(-c4ccc5c(c4)OP(=O)(O)O5)c(=O)c3c1O)C=CC(C)(C)O2 ; "or" ; CC(C)=CCc1c2c(c3occ(C4=CC(=O)C(=O)C=C4)c(=O)c3c1O)C=CC(C)(C)O2]

2. The compound of claim 1, wherein the compound is

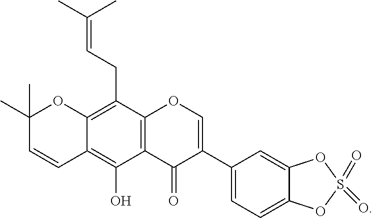 [SMILES: CC(C)=CCc1c2c(c(O)c3c(=O)c(-c4ccc5c(c4)OS(=O)(=O)O5)coc13)C=CC(C)(C)O2]

3. The compound of claim 1, wherein the compound is

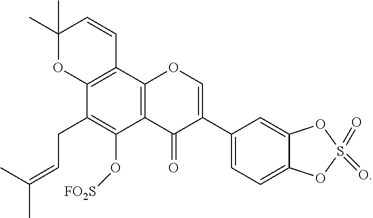

4. The compound of claim 1, wherein the compound is

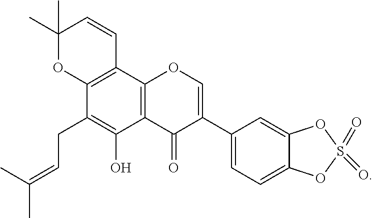 [SMILES: CC(C)=CCc1c2c(c3occ(-c4ccc5c(c4)OS(=O)(=O)O5)c(=O)c3c1O)C=CC(C)(C)O2]

5. The compound of claim 1, wherein the compound is

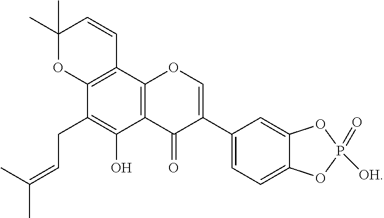 [SMILES: CC(C)=CCc1c2c(c3occ(-c4ccc5c(c4)OP(=O)(O)O5)c(=O)c3c1O)C=CC(C)(C)O2]

6. The compound of claim 1, wherein the compound is

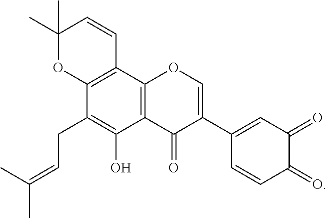 [SMILES: CC(C)=CCc1c2c(c3occ(C4=CC(=O)C(=O)C=C4)c(=O)c3c1O)C=CC(C)(C)O2]

7. A therapeutically effective composition, comprising a compound of claim 1 and a pharmaceutically acceptable carrier.

8. The therapeutically effective composition of claim 7, wherein the compound is

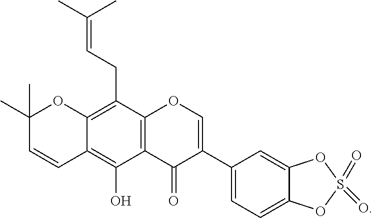 [SMILES: CC(C)=CCc1c2c(c(O)c3c(=O)c(-c4ccc5c(c4)OS(=O)(=O)O5)coc13)C=CC(C)(C)O2]

9. The therapeutically effective composition of claim 7, wherein the compound is

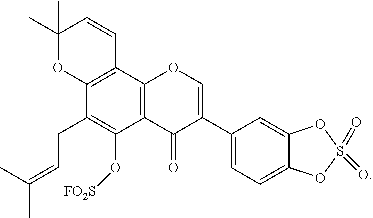

10. The therapeutically effective composition of claim 7, wherein the compound is

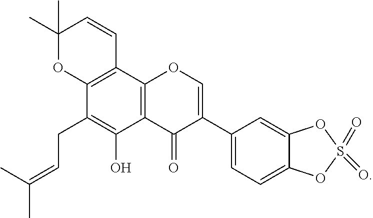 [SMILES: CC(C)=CCc1c2c(c3occ(-c4ccc5c(c4)OS(=O)(=O)O5)c(=O)c3c1O)C=CC(C)(C)O2]

11. The therapeutically effective composition of claim 7, wherein the compound is

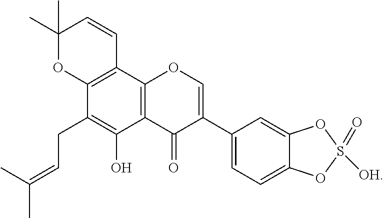

12. The therapeutically effective composition of claim 7, wherein the compound is

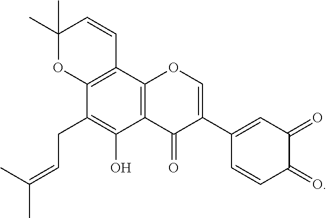 [SMILES: CC(C)=CCc1c2c(c3occ(C4=CC(=O)C(=O)C=C4)c(=O)c3c1O)C=CC(C)(C)O2]

13. (canceled)

14. A method of inhibiting a Crk-mediated signaling pathway, comprising exposing a sample or a subject to a compound of claim 1, or a therapeutically effective composition thereof.

15. The method of claim 14, wherein the sample is an in vitro or ex vivo sample.

16. The method of claim 14, wherein the compound inhibits tumor cell line proliferation and does not suppress immune cell function.

17. A method of augmenting immune cell response in a subject having, or suspected of having, a chronic viral infection or a cancer, comprising administering the subject to a compound of claim 1, or a therapeutically effective composition thereof.

18. The method of claim 17, wherein the compound is

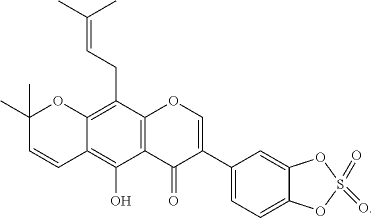 [SMILES: CC(C)=CCc1c2c(c(O)c3c(=O)c(-c4ccc5c(c4)OS(=O)(=O)O5)coc13)C=CC(C)(C)O2]

19. The method of claim 17, wherein the compound is

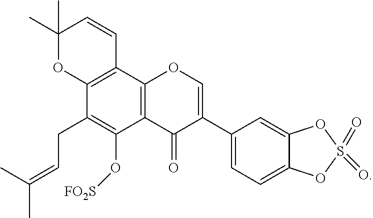

20. The method of claim 17, wherein the compound is

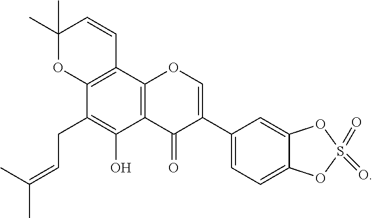 [SMILES: CC(C)=CCc1c2c(c3occ(-c4ccc5c(c4)OS(=O)(=O)O5)c(=O)c3c1O)C=CC(C)(C)O2]

21. The method of claim 17, wherein the compound is

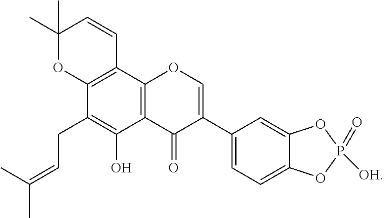 [SMILES: CC(C)=CCc1c2c(c3occ(-c4ccc5c(c4)OP(=O)(O)O5)c(=O)c3c1O)C=CC(C)(C)O2]

22. The method of claim 17, wherein the compound is 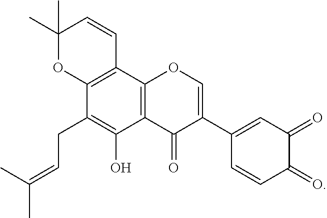 [SMILES: CC(C)=CCc1c2c(c3occ(C4=CC(=O)C(=O)C=C4)c(=O)c3c1O)C=CC(C)(C)O2]
